(19) United States
(12) Patent Application Publication
(10) Pub. No.: US 2017/0357751 A1
(43) Pub. Date: Dec. 14, 2017

(54) COMPUTER-IMPLEMENTED EVALUATON OF DRUG SAFETY FOR A POPULATION

(71) Applicant: Cipherome, Inc., Cupertino, CA (US)

(72) Inventors: Kye H. LEE, Seoul (KR); Paul J. PARK, Reno, NV (US)

(21) Appl. No.: 15/688,599

(22) Filed: Aug. 28, 2017

Related U.S. Application Data
(63) Continuation of application No. PCT/US16/66230, filed on Dec. 12, 2016.
(60) Provisional application No. 62/266,578, filed on Dec. 12, 2015.

(51) Int. Cl. G06F 19/18; G06F 19/00; G06F 19/24; C12Q 1/68

(52) U.S. Cl. CPC G06F 19/18; C12Q 1/6883; G06F 19/363; G06F 19/24; C12Q 1/68

(57) ABSTRACT

A computer-implemented drug evaluation method and system provides for evaluating safety of a drug or a drug group by performing certain computations associated with gene sequence variation information of individuals within a population. The system calculates various scores for individual within a population and ultimately combines the scores in determining safety of the drug across the population. The drug evaluation method and a system can further be configured for identifying individuals having a high-risk of side effects to a drug or a drug group. The drug evaluation provides universal drug safety information based on gene sequence variation information without the need to identify specific genetic markers for each drug.

### 1. CROSS-REFERENCE

This application is a continuation of International Application No. PCT/US16/66230, filed Dec. 12, 2016, which claims the benefit of U.S. Provisional Application No. 62/266,578 filed on Dec. 12, 2015, each of which is incorporated herein by reference in its entirety.

### 2. FIELD OF THE INVENTION

The present invention generally relates to a computer-implemented drug safety evaluation, and more specifically a computer-implemented evaluation of drug safety across a population of individuals.

### 3. BACKGROUND

Clinical studies are usually conducted on the assumption that there is no significant variation in the genes associated with absorption, metabolism, action and excretion of a drug in a population. As a result, a subpopulation with a high pharmacogentic risk may be under-represented in small-scale clinical studies involving 2000-3000 subjects, and not all the side effects of a drug are discovered through clinical studies. In fact, there have been a number of drugs which were once released in the market but later withdrawn because of side effects which had not been found through the clinical studies. Previously, high-risk subpopulations have generally not been identified when conducting or analyzing clinical studies.

Genetic analysis enables prediction of response to drugs or chemicals. For example, genetic differences (e.g., genetic polymorphism of enzymes involved in drug metabolism) have been associated with efficacy or side effects of a number of drugs. The efficacy or side effects of a drug may be different among individuals because drug metabolism can be slower or faster depending on the particular genetic variations of the individuals.

Researchers have carried out studies in this regard to identify drug responses associated with genetic variations, in addition to identifying the severity of diseases to be treated, drug-drug interactions, and also the age, nutritional condition, and liver/kidney function of a patient, along with environmental factors for a patient, such as climate or food. For example, researchers have examined efficacy of certain drugs in patients with chronic conditions by evaluating the effect of polymorphisms in select candidate genes on the response of patients to the drugs. In addition, pharmacogenetics or pharmacogenomics based studies on the interrelationship between genomic information such as a single-nucleotide polymorphism (SNP) as markers and drug response/side effects, etc. have been done.

However, it has been difficult to find such genetic markers for each drug that allows prediction of response to each drug. Responses to drugs usually result from a complex interplay between genetic variations in individuals' genome sequences, the drug, and various other factors which are difficult to control or be identified. A drug associated with a larger variability of related genes is more likely to cause diverse drug responses. Prior work does not provide useful and reliable drug information for various subpopulations beyond methods based on observational studies of a population using markers, such as single-nucleotide polymorphisms.

### 4. SUMMARY

Computer-implemented drug safety evaluation is used for predicting safety of a drug without requiring identifying of genetic markers. A drug safety evaluation system infers protein damage by analyzing gene sequence variation information of individuals. In some embodiments, the system computes drug safety scores of individuals based thereon. The evaluation system further provides a way of evaluating drug safety by analyzing gene sequence variation information and individual drug safety scores of each individual within a given population. Furthermore, in some embodiments, the system calculates a population drug safety score indicating drug safety to the population. Thus, the system enables prediction of population response to a drug without requiring the need to identify genetic markers. It further allows prediction of a subpopulation having a high risk of side effects to the drug.

The evaluation methods and systems of the present invention are applicable to a whole range of drugs for which protein information involved in the pharmacodynamics or pharmacokinetics can be acquired with respect to metabolism, effects, side effects, etc. of the drugs. With conventional pharmacogenomics studies, it is required that the study be conducted on each drug-gene pair, yet it is practically impossible to study all the numerous drug-gene pairs because the number of pairs increases in proportion to the product of the number of drugs and the number of gene markers. Thus, these conventional studies have not been able to provide sufficient data, and a high statistical error results from the selection of study subjects and the difference between population groups. In contrast, the evaluation systems and methods described here are directly applicable to customized drug therapy, and thus data of nearly all drug-gene pairs can be acquired. In addition, the method can be applied by applying the difference between population groups when calculating the population drug safety scores and the individual drug safety score distribution curve.

Some embodiments of the present invention relate to a computer-implemented method for evaluating safety of a drug, comprising the steps of: (1) obtaining, by an evaluation system, gene sequence variation information for each of a plurality of individuals within a population, wherein the gene sequence variation information is related to one or more genes associated with pharmacodynamics or pharmacokinetics of the drug; (2) calculating, by the evaluation system, a protein damage score for each of the plurality of individuals within the population using the gene sequence variation information; (3) calculating, by the evaluation system, an individual drug safety score for each of the plurality of individuals within the population based on the protein damage score to generate a set of individual drug safety scores; and (4) determining, by the evaluation system, safety of the drug for the population based on the set of individual drug safety scores.

In some embodiments, the step of determining safety of the drug comprises obtaining a curve representing the set of individual drug safety scores. In some embodiments, the step further comprises calculating an area under the curve (AUC), a standardized area under the curve (S-AUC), an area upper the curve (AUPC), or a standardized area upper the curve (S-AUPC).

In some embodiments, the method for evaluating safety of a drug further comprises the step of calculating a population drug safety score using the following Equation:

$${S}_{p} ( d 1 , … , dn ) = \frac{1}{N} ( A U {C}_{d} ) = 1 - \frac{1}{N} ( A U P {C}_{d} )$$

wherein Sp is the population drug safety score for the population, d1-n is an individual drug safety score of an i-th individual (from 1 to n) within the population, AUCd is the area under the curve for the drug d, AUPCd is the area upper the curve for the drug d, and N or n is the number of individuals within the population.

In some embodiments, the step of determining safety of the drug comprises identifying individuals having an individual drug safety score below or above a threshold value. In some embodiments, the threshold value (T) is calculated by the Equation:

$$T = μ - κ \sqrt{\frac{1}{n} {\sum }_{i = 1}^{n} {( {d}_{i} - μ )}^{2}}$$

wherein T is a rational number satisfying 0<T<1, di is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores.

In some embodiments, the threshold value (T) is determined based on the shape of the curve. In some embodiments, the threshold value (T) is calculated based on the change in the slope of the curve. In some embodiments, the threshold value (T) is determined by comparing the curve with a different curve corresponding to a different drug having similar pharmacodynamics or pharmacokinetics or a different drug previously identified to be unsafe.

In some embodiments, the threshold value (T) ranges from 0.1 to 0.5, from 0.2 to 0.4, or from 0.25 to 0.35, or is 0.3. In some embodiments, the method for evaluating safety of a drug further comprises the step of providing a list of the individuals having an individual drug safety score below a threshold value or above a threshold value.

In some embodiments, the step of determining safety of the drug further comprises: calculating the number or the ratio of individuals having an individual drug safety score below the threshold value within the population. In some embodiments, the method further comprises the step of calculating a population drug safety score of the population, wherein the population drug safety score is related to the number or the ratio of individuals having a drug safety score below the threshold value within the population.

In some embodiments, the step of determining safety of the drug comprises calculating a mean of individual drug safety scores of multiple individuals within the population, wherein the mean is calculated using one or more algorithms selected from the group consisting of a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication, a weighted multiplication, or a combination thereof.

In some embodiments, the method of evaluating safety of a drug further comprises the step of providing a population drug safety score of the population calculated by the following Equation:

$${S}_{d} ( {d}_{1} , … , {d}_{n} ) = \frac{1}{n} {\sum }_{i = 1}^{n} {d}_{i}$$

wherein Sd is the population drug safety score, di or d1-n is an individual drug safety score of an individual within the population (from 1 to n), and n is the number of individuals within the population for which the individual drug safety score is obtained.

In some embodiments, the gene sequence variation information is information related to substitution, addition, or deletion of a nucleotide within the exon of the gene. In some embodiments, the substitution, addition, or deletion of the nucleotide results from breakage, deletion, duplication, inversion or translocation of a chromosome.

In some embodiments, the method of evaluating safety of a drug further comprises the step of obtaining a gene sequence variation score from the gene sequence variation information, using one or more algorithm selected from the group consisting of: SIFT (Sorting Intolerant From Tolerant), PolyPhen, PolyPhen-2 (Polymorphism Phenotyping), MAPP (Multivariate Analysis of Protein Polymorphism), Logre (Log R Pfam E-value), Mutation Assessor, Condel, GERP (Genomic Evolutionary Rate Profiling), CADD (Combined Annotation-Dependent Depletion), MutationTaster, MutationTaster2, PROVEAN, PMuit, CEO (Combinatorial Entropy Optimization), SNPeffect, fathmm, MSRV (Multiple Selection Rule Voting), Align-GVGD, DANN, Eigen, KGGSeq, LRT (Likelihood Ratio Test), MetaLR, MetaSVM, MutPred, PANTHER, Parepro, phastCons, PhD-SNP, phyloP, PON-P, PON-P2, SiPhy, SNAP, SNPs&GO, VEP (Variant Effect Predictor), VEST (Variant Effect Scoring Tool), SNAP2, CAROL, PaPI, Grantham, SInBaD, VAAST, REVEL, CHASM (Cancer-specific High-throughput Annotation of Somatic Mutations), mCluster, nsSNPAnayzer, SAAPpred, HanSa, CanPredict, FIS and BONGO (Bonds ON Graphs).

In some embodiments, the gene sequence variation score is used to calculate the protein damage score or the individual drug safety score.

In some embodiments, the method of evaluating safety of a drug further comprises the step of obtaining a plurality of gene sequence variation scores from the gene sequence variation information, wherein the gene sequence variation information relates to substitution, addition, or deletion of a plurality of nucleotides within the gene. In some embodiments, the protein damage score is calculated as a mean of the plurality of gene sequence variation scores. In some embodiments, the mean is calculated using one or more algorithms selected from the group consisting of: a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication and a weighted multiplication.

In some embodiments, the protein damage score is calculated by the following Equation:

$${S}_{g} ( {v}_{1 , … ,} {v}_{n} ) = {( \frac{1}{n} {\sum }_{i = 1}^{n} {v}_{i}^{p} )}^{\frac{1}{p}}$$

wherein Sg is a protein damage score of a protein encoded by the gene g, n is the number of the plurality of nucleotides corresponding to the plurality of gene sequence variation scores, vi is a gene sequence variation score corresponding to an i-th gene sequence variation, and p is a non-zero real number.

In some embodiments, the protein damage score is calculated by the following Equation:

$${S}_{g} ( {v}_{1 , … ,} {v}_{n} ) = {( {\prod }_{i = 1}^{n} {v}_{i}^{{w}_{i}} )}^{1 / {\sum }_{i = 1}^{n} {w}_{i}} ,$$

wherein Sg is a protein damage score of a protein encoded by the gene g, n is the number the plurality of nucleotides corresponding to the plurality of gene sequence variation scores, vi is a gene sequence variation score corresponding to an i-th gene sequence variation, and wi is a weighting assigned to the gene sequence variation score vi of the i-th gene sequence variation.

In some embodiments, the method of evaluating safety of a further comprises the step of obtaining protein damage scores, wherein each of the protein damage scores corresponds to each of the plurality of proteins involved in the pharmacodynamics or pharmacokinetics of the drug. In some embodiments, the individual drug safety score is calculated as a mean of the protein damage scores. In some embodiments, the mean is calculated using one or more algorithm selected from the group consisting of: a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication and a weighted multiplication.

In some embodiments, the individual drug safety score is calculated by the following Equation:

$${S}_{d} ( {g}_{1 , … ,} {g}_{n} ) = {( \frac{1}{n} {\sum }_{i = 1}^{n} {g}_{i}^{p} )}^{\frac{1}{p}}$$

wherein Sd is an individual drug safety score of a drug d, n is the number of proteins encoded by one or more genes involved in the pharmacodynamics or pharmacokinetics of the drug d, gi is a protein damage score of the protein encoded by one or more genes involved in the pharmacodynamics or pharmacokinetics of the drug d, and p is a non-zero real number.

In some embodiments, the individual drug safety score is calculated by the following Equation:

$${S}_{d} ( {g}_{1 , … ,} {g}_{n} ) = {( {\prod }_{i = 1}^{n} {g}_{i}^{{w}_{i}} )}^{1 / {\sum }_{i = 1}^{n} {w}_{i}}$$

wherein Sd is a drug score of the drug d, n is the number of proteins encoded by one or more genes involved in the pharmacodynamics or pharmacokinetics of the drug d, gi is a protein damage score of the protein encoded by one or more genes involved in the pharmacodynamics or pharmacokinetics of the drug d, and wi is a weighting assigned to the protein damage score gi of the protein encoded by one or more genes involved in the pharmacodynamics or pharmacokinetics of the drug d.

Some embodiments of the present invention relates to a computer-implemented method of evaluating safety of a drug group, comprising the steps of: (1) identifying drugs that belong to the drug group; (2) obtaining a population drug safety score for each of the drugs, thereby generating a set of population drug safety scores, wherein the population drug safety score is calculated by the methods described above; and (3) analyzing the set of population drug safety scores.

In some embodiments, the method of evaluating safety of a drug group further comprises the step of determining an order of priority among the drugs based on the analysis.

In some embodiments, the step of analyzing the set of population drug safety scores comprises calculating a mean of the set of population drug safety scores, wherein the mean is calculated using one or more algorithms selected from the group consisting of a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication, a weighted multiplication, or a combination thereof.

In some embodiments, the step of identifying drugs that belong to the drug group is performed based on (i) known drug classification methods, (ii) symptoms known to be treatable by the drugs, (iii) a chemical property of the drugs, (iv) an absorption or excretion mechanism of the drugs, or (v) a target of the drugs.

Some embodiments of the present invention relates to a method of evaluating safety of a drug to a subject, comprising the steps of (1) obtaining gene sequence variation information of the subject, wherein the gene sequence variation information is related to one or more genes associated with pharmacodynamics or pharmacokinetics of the drug; (2) obtaining a protein damage score of the subject using the gene sequence variation information; (3) obtaining a subject drug safety score of the subject based on the protein damage score; and (4) determining safety of the drug for the subject by comparing the subject drug safety score with the set of individual drug safety scores obtained by any of the methods described above.

In some embodiments, the step of determining safety of the drug to the subject comprises the step of determining a position of the subject drug safety score within the set of individual drug safety scores.

In some embodiments, the step of determining safety of the drug to the subject comprises the steps of: (1) drawing a curve with the set of individual drug safety scores; (2) obtaining an area under the curve (AUC), a standardized area under the curve (S-AUC), an area upper the curve (AUPC), or a standardized area upper the curve (S-AUPC); and (3) comparing the subject drug safety score with the AUC, S-AUC, AUPC, or S-AUPC.

In some embodiments, the step of determining safety of the drug to the subject comprises the steps of: (1) obtaining a threshold value (T) corresponding to the set of individual drug safety scores, wherein the threshold value (T) is calculated by the Equation:

$$T = μ - κ \sqrt{\frac{1}{n} {\sum }_{i = 1}^{n} {( {d}_{i} - μ )}^{2}}$$

wherein di is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores; and (2) comparing the subject drug safety score with the threshold value (T).

In some embodiments, the step of determining safety of the drug to the subject comprises the steps of: (1) obtaining a population drug safety score of the population calculated by the Equation:

$${S}_{d} ( {d}_{1} , … , {d}_{n} ) = \frac{1}{n} {\sum }_{i = 1}^{n} {d}_{i}$$

wherein Sd is the population drug safety score of the population, di is an individual drug safety score of an i-th individual within the population (from 1 to n), and n is the number of individuals within the population; and (2) comparing the subject drug safety score with the population drug safety score (Sd).

In some embodiments, the method of evaluating safety of a drug for a subject further comprises the step of prescribing the drug based on the safety of the drug to the subject.

Some embodiments of the present invention also relates to a computer-readable medium comprising stored instructions, wherein the instructions when executed by a processor cause the processor to perform any of the methods described above. In some embodiments, the instructions further cause the processor to provide a report related to safety of the drug, safety of the drug group or safety of the drug to the subject.

Some embodiments of the present invention relates to a system for evaluating safety of a drug, comprising: (1) the computer-readable medium described above; and (2) an output unit providing the report about the safety of the drug. In some embodiments, the output unit provides the report by email, SMS messaging, web posting, phone call, electronic messaging, uploading or downloading. In some embodiments, the system further comprises a database to search for or retrieve information about one or more genes associated with pharmacodynamics or pharmacokinetics of the drug.

### 5. BRIEF DESCRIPTION OF THE DRAWINGS

FIG. 1 is a schematic diagram of a computing environment including a system for providing drug safety information using gene sequence variation of individuals within a population according to an exemplary embodiment of the present invention.

FIG. 2 is a flowchart illustrating each step of various methods for evaluating drug safety using gene sequence variation of individuals within a population according to an exemplary embodiment of the present invention.

FIG. 3 schematically illustrates a method for calculating a gene sequence variation score (V₁₋₁₃), a protein damage score corresponding to Gene_{1-d}. (S_{g(a)}, S_{g(b)}, S_{g(c)}, S_{g(d)}, . . . ), an individual drug safety score (S_{d(k)}, S_{d(j)}, . . . ), and a population drug safety score (Sₚ). FIG. 3 further describes a method of predicting drug safety for an individual by comparing the individual's drug safety score (S_{d(k)} for H1 and S_{d(j)} for H1) and the individual drug safety score distribution curve corresponding to each drug.

FIG. 4A provides three distribution curves of individual drug safety scores from 2504 individuals (provided by the 1000 Genomes Project, Phase III), each corresponding to a drug previously withdrawn from the market according to DrugBank, the UN and the EMA. (top line with triangles for disoyramide; middle line with circles for procainamide; and bottom line with rectangles for quinidine, respectively)

FIG. 4B provides a bar graph representing an area under the curve (AUC) for each drug. AUC for disopyramide is measured as 1−α, AUC for procainamide is measured as 1−(α+(3), and AUC for quinidine is measured as 1−(α+(3+y).

FIG. 4C provides a graph with three bars, each representing individual drug safety scores corresponding to the bottom 30% or 70% in the distribution curve of individual drug safety scores.

FIGS. 5A-5I provide histograms presenting withdrawal rates of various drugs based on their population drug safety scores. X-axis provides 10 score sections for different ranges of population drug safety scores between 0 and 1 and y-axis provides average withdrawal rates of the drugs corresponding to the respective score sections.

FIGS. 6A-6F provide a distribution curve of individual drug safe scores for Rosuvastatin, each corresponding to one of five race groups—FIG. 6B is for American (AMR), FIG. 6C is for European (EUR), FIG. 6D is for East Asian (EAS), FIG. 6E is for African (AFR), FIG. 6F is for South Asian (SAS), and FIG. 6A is for a combination of all five race groups. The arrows in FIG. 6A indicate rankings of individuals having 0.3 as an individual drug safety score. The arrows in FIGS. 6B-6F indicate individual drug safety scores of individuals having the same ranking (30) of individual drug safety scores within each race group.

FIGS. 7A-7F provide a distribution curve of individual drug safety scores for six different drugs classified as antipsychotics by the Anatomical Therapeutic Chemical (ACT) Classification System provided by the WHO. FIG. 7A is for oxazepam, FIG. 7B is for bromazepam, FIG. 7C is for fludiazepam, FIG. 7D is for ketazolam, FIG. 7E is for prazepam, and FIG. 7F is for tofisopam.

FIGS. 8A-8F provide a distribution curve of individual drug safety scores for six different drugs classified as lipid modifying agents by the Anatomical Therapeutic Chemical (ACT) Classification System provided by the WHO. FIG. 8A is for simvastatin, FIG. 8B is for fluvastatin, FIG. 8C is for atorvastatin, FIG. 8D is for pravastatin, FIG. 8E is for rosuvastatin, and FIG. 8F is for pitavastatin.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The term “pharmacokinetics (PK) or pharmacokinetic parameter” used in the present invention refers to characteristics of a drug involved in absorption, migration, distribution, conversion and excretion of the drug in the body for a particular time period and includes the volume of distribution (Vd), clearance rate (CL), bioavailability (F) and absorption rate coefficient (kₐ) of a drug, or maximum plasma concentration (Cₘₐₓ), time point of maximum plasma concentration (Tₘₐₓ), area under the curve (AUC) regarding a change in plasma concentration for a certain time period, etc. The term “pharmacodynamics or pharmacodynamic parameter” used in the present invention refers to characteristics involved in physiological and biochemical behaviors of a drug with respect to the body and mechanisms thereof, i.e., responses or effects in the body caused by the drug.

The term “pharmacokinetic parameter of an enzyme protein of a drug” used in the present invention includes Vₘₐₓ, Kₘ, K_{cat}/Kₘ, etc. Vₘₐₓ is the maximum enzyme reaction rate when a substrate concentration is very high, and Kₘ is the substrate concentration that causes the reaction to reach ½ Vₘₐₓ. Kₘ may be regarded as affinity between the corresponding enzyme and the corresponding substrate. As the Kₘ is decreased, a bonding force between the corresponding enzyme and the corresponding substrate is increased. K_{cat}, which is called the turnover number of an enzyme, refers to the number of substrate molecules metabolized for 1 second in each enzyme active site when the enzyme is activated at a maximum rate, and means how fast the enzyme reaction actually occurs.

The term “sequence variation information” used in the present invention refers to information related to substitution, addition or deletion of a nucleotide in a gene. The substitution, addition or deletion can be located in an exon or an intron of the gene, or other regulatory sequence.

The term “gene sequence variation score” used in the present invention refers to a numerical score of a degree of the individual gene sequence variation, when the gene sequence variation is found in the exon region of the gene encoding the protein, that causes an amino acid sequence variation (substitution, addition or deletion) of a protein encoded by a gene or a variation in transcription regulation and thus causes a significant change in the protein expression. The gene sequence variation score can be calculated considering a degree of evolutionary conservation of amino acids in a genome sequence, a degree of an effect of a physical characteristic of modified amino acids on the structure or function of the corresponding protein, etc.

The term “protein damage score” used in the present invention refers to a score calculated by based on gene sequence variation scores. If there is a single significant sequence variation in the gene region encoding the protein, a gene sequence variation score is identical to a protein damage score. If there are two or more gene sequence variations encoding the protein, a protein damage score is calculated as a mean of gene sequence variation scores calculated for the respective variations.

The term “individual drug safety score” used in the present invention refers to a value calculated with respect to a particular drug and an individual by finding out one or more target proteins involved in the pharmacodynamics or pharmacokinetics of the drug, such as an enzyme protein involved in drug metabolism, a transporter protein or a carrier protein. The individual drug safety score can be calculated based on protein damage scores of one or more genes encoding proteins involved in the pharmacodynamics or pharmacokinetics of the drug with respect to the individual.

The term “population drug safety score” used in the present invention refers to a value calculated based on individual drug safety scores of individuals belonging to a particular population for a drug. The population drug safety score can be obtained by calculating the area under the curve (AUC) of an individual drug safety score distribution curve and dividing the AUC by the number of the individuals constituting the population (S-AUC). Similarly, the value obtained by dividing the area upper the individual drug safety score distribution curve by the number of the individuals constituting the population is called a standardized area upper the curve (S-AUPC) and it can be used as the population drug safety score. In some embodiments, the population drug safety score can be obtained by calculating the mean of individual drug safety scores of individuals belonging to a particular population.

The term “individual drug safety score distribution curve” or “distribution curve of individual drug safety scores” used in the present invention refers to a plot of the distribution of individual drug safety scores of individuals within a particular population. It includes a line graph obtained by plotting the individual drug safety scores from lower to higher scores, a density curve plotted using a density estimation function, a histogram, etc., although not being limited thereto.

The term “drug safety threshold score” used in the present invention refers to a specific drug safety score allowing for determining a high-risk subpopulation using individual drug safety scores of individuals within a population or their distribution curve. Individuals with an individual drug safety score below a threshold score for a particular drug have more variations causing damage in the proteins associated with the pharmacodynamics or pharmacokinetics of the drug than individuals with an individual drug safety score above the threshold score.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 6.3. Methods for Carrying Out Invention

6.3.1. A System for Evaluating Drug Safety

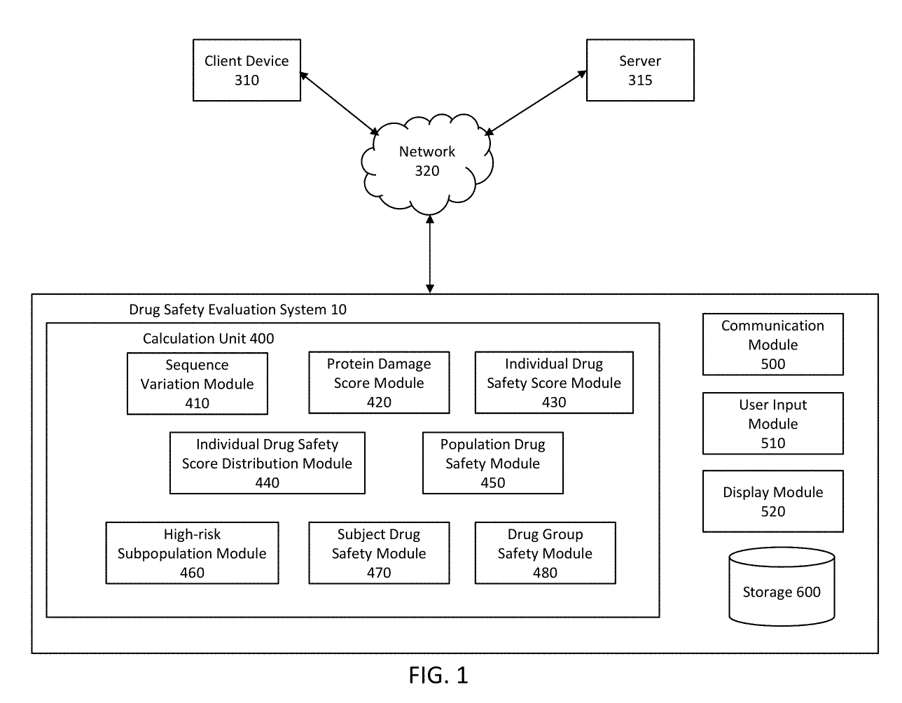

FIG. 1 is a schematic diagram of a computing environment including a system for evaluating drug safety using gene sequence variation information of individuals within a population according to an exemplary embodiment of the present invention. The computing environment includes one or more client devices 310, one or more servers 315, and a drug safety evaluation system 10 all connected through a network 320.

The client device 310 is a computing device capable of receiving user input as well as transmitting and/or receiving data via the network 320. In one embodiment, a client device 310 is a conventional computer system, such as a desktop or laptop computer. Alternatively, a client device 310 may be a device having computer functionality, such as a personal digital assistant (PDA), a mobile telephone, a smartphone or another suitable device. A client device 310 is configured to communicate via the network 320. In one embodiment, a client device 310 executes an application allowing a user of the client device 310 to interact with the drug safety evaluation system 10. For example, a client device 310 executes an application to enable interaction between the client device 310 and the drug safety evaluation system 10 via the network 320. In some embodiments, the client device 310 allows the user to provide inputs to the drug safety evaluation 10 and the user can also receive information from the drug safety evaluation system 10 displayed on a user interface of the client device 310. As one example, the client device 310 might be operated by a pharmaceutical company or a research institution interested in performing a study or obtaining information about drug safety for a particular drug of interest in population of interest. In this example, the company or institution uses the client device 310 to request a drug safety evaluation, and in some cases to provide data about the drug and the population to the drug safety evaluation system 10. In some embodiments, the client device 310 is used for providing gene sequence information for a number of individuals within the population of interest. The drug safety evaluation system 10 performs the evaluation and provides results to the client device 310 with regard to safety of the drug for the population. The results can be displayed in a user interface on the client device 310.

The network 320 includes any combination of local area and/or wide area networks, using both wired and/or wireless communication systems. In one embodiment, the network 320 uses standard communications technologies and/or protocols. For example, the network 320 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 320 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 320 may be represented using any suitable format, such as hypertext markup language (HTML) or extensible markup language (XML). In some embodiments, all or some of the communication links of the network 320 may be encrypted using any suitable technique or techniques.

The server 315 is a computing device capable of transmitting and/or receiving data via the network 320. The server 315 can also be a collection of servers. The server 315 can be associated with the drug safety evaluation system 10 and can act as storage or can transmit/receive data from the system. In some embodiments, the server 315 can be an outside system separate from the drug safety evaluation system 10 that sends and receives data to the system 10. For example, the server 315 could be owned by a laboratory that sends sequence information of the patient to the system 10. In some embodiments, the server is a means for providing an access to database with respect to a drug, a gene variation or a drug-protein relation and is connected to drug safety evaluation system 10 through the communication module 500 so as to exchange various kinds of information. In some embodiments, one or more servers 315 are operated by a party interested in or requesting the drug safety evaluation for a population and drug of interest.

The drug safety evaluation system 10 may include a variety of modules/components, including calculation unit 400 having a sequence variation module 410, a protein damage score module 420, an individual drug safety score module 430, an individual drug safety score distribution module 440, a population drug safety module 450, a high-risk subpopulation module 460, a subject drug safety module 470, and a drug group safety module 480. The drug safety evaluation system 10 may also include a communication module 550, a user input module 510, a display module 520, and a storage module 600. In other embodiments, the system 10 may include additional, fewer, or different modules for various applications. A number of the modules in the calculation unit 400 are configured for computing certain scores associated with the drug safety evaluation. The modules are introduced briefly first and the scores are described in more detail after this introduction.

The sequence variation module 410 of the calculation unit 400 is configured to calculate one or more gene sequence variations for the patient that are involved in the pharmacodynamics or pharmacokinetics of the drug or drug group. These computed sequence variations are used to evaluate drug safety. In some embodiments, the sequence variation module 410 simply obtains or receives this individual sequence variation information for individuals within a population. The individuals can directly provide the sequence variation information to which the individuals have access, a third party having sequence variation information, such as a drug maker or a company conducting clinical studies, can provide the sequence variation information, or this information can be received by the module 500 from a laboratory that conducted the sequence variation analysis to determine the individual sequence variations. In some cases, the raw sequence data is provided to the module 500 and the module determines the variations.

In one embodiment, the sequence variation module 410 calculates a gene sequence variation score, which is described in more detail below. The gene sequence variation score can be calculated for each individual within the population of interest. The score indicates the degree of the individual genome sequence variation that causes an amino acid sequence variation (substitution, addition, or deletion) of a protein encoded by a gene or a transcription control variation, and thus causes a significant change or damage to a structure and/or function of the protein when the genome sequence variation is found in an exon region of the gene encoding the protein.

The protein damage score module 420 of the calculation unit 400 is configured to calculate an individual protein damage score for an individual based on the individual's gene sequence variation information. The protein damage score is calculated by summarizing or combining gene sequence variation scores (or otherwise combining another quantitation of the gene sequence variation) to get an indication of damage or modification to the protein of the individual that results from the sequence variations. In embodiments in which there is no protein damage score, this module may not exist or may not be used.

The individual drug safety score module 430 of the calculation unit 400 is configured to calculate an individual drug safety score for a drug by associating the individual protein damage score with a drug-protein relation. In embodiments in which there is no drug safety score, this module may not exist or may not be used.

The individual drug safety score distribution module 440 of the calculation unit 400 is configured to provide a distribution curve of individual drug safety scores. In embodiments in which there is no individual drug safety score curve, this module may not exist or may not be used.

The population drug safety module 450 of the calculation unit 400 is configured to evaluate safety of a drug for a population. In some embodiments, the module 450 calculates a population drug safety score, which is described in more detail below.

The high-risk subpopulation module 460 of the calculation unit 400 is configured to identify individuals having high-risk with a drug, such as a high risk that the drug will cause adverse side effects in the subpopulation. In embodiments in which there is no identification of high-risk subpopulation, this module may not exist or may not be used.

The subject drug safety module 470 of the calculation unit 400 is configured to evaluate safety of a drug for a subject. For example, the module 460 can provide an evaluation of the drug for a single individual as opposed to a population of individuals. In some embodiments, the module communicates data to different modules of the calculation unit 400 such as the individual drug safety score module 430, individual drug safety score distribution module 440, and population drug safety score module 490, to evaluate drug safety for the subject. In embodiments in which there is no evaluation of safety of a drug for a subject, this module may not exist or may not be used.

The drug group safety module 480 of the calculation unit 400 is configured to evaluate safety of a drug group. In some embodiments, the module 480 has access to information about drugs or drug groups. This information may be accessed from storage associated with the evaltuation system or may be provided by another entity, such as the client device or server. In embodiments in which there is no evaluation of safety of a drug group, this module may not exist or may not be used.

The user input module 510 is configured to receive as input information about drugs or drug groups from the user, or is configured to access storage 600 that stores information about the drugs or drug groups effective in treating a specific disease and extract relevant information, and this can thereby be used to calculate and provide an individual and population drug safety score of the drug. The user input module 510 can also receive other inputs from the user, such as information about the population such as race, gender, age, affected diseases or symptoms. The user input module 510 can also receive other information that can be used for evaluation of drug safety.

The display module 520 is configured to display or to provide to a client device for display the values calculated by the respective modules or a calculation process for determining drug safety and information as a ground for the calculation or determination.

The communication module 500 controls communication between the drug safety evaluation system 10 and outside entities, such as communication over the network 320. For example, the module 500 can manage the communication with a lab to receive sequence variation information.

The storage 600 can be any database or data storage (or knowledge base) or collection of databases that can store information that can be accessed by components of the system 10. The database may be directly installed in the server and may also be connected to various life science databases accessible via the Internet depending on the purpose. In the system according to the present invention, the database or a server including access information, the calculated information, and the user interface connected thereto may be used as being linked to one another.

In some embodiments, if new pharmacological/biochemical information regarding a drug-protein relation is produced, the system can be immediately updated so as to be used for further improved personalization of drug selection. In an exemplary embodiment, when the database or knowledge base is updated, the gene sequence variation information, gene sequence variation score, protein damage score, individual drug safety score, population drug safety score and the information as grounds for the calculation thereof stored in the respective modules are updated.

The method according to the present invention can be implemented by hardware, firmware, software, or combinations thereof. If the method is implemented by software, a storage medium may include any storage or transmission medium readable by a device such as a computer. For example, the computer-readable medium may include a ROM (read only memory); a RAM (random access memory); a magnetic disc storage medium; an optical storage medium; a flash memory device; and other electric, optical or acoustic signal transmission medium.

In some embodiments, the present invention provides a computer-readable medium comprising an execution module which executes a processor, the processor performing operations comprising: a step of acquiring one or more gene sequence variation information associated with the pharmacodynamics or pharmacokinetics of a particular drug or drugs from genome sequence information of individuals; a step of calculating protein damage scores of individuals using the gene sequence variation information; and a step of calculating individual drug safety scores for individuals and a population drug safety score for a population. The processor may further comprise: determining the order of priority among drugs applicable to an individual by using the above-described individual drug safety score and/or population drug safety score; or determining whether or not to use the drugs applicable to the individual by using the above-described individual drug safety score and/or population drug safety score.

In another aspect, the present invention relates to a system for providing drug safety information using gene sequence variation information of individuals within a population comprising: a database to search for or retrieve information associated with genes or proteins related with a drug or drugs applied to individuals; a communication unit which can access the database; a sequence variation module which calculates one or more gene sequence variation information associated with the pharmacodynamics or pharmacokinetics of the drug or drugs based on the information; a protein damage score module which calculates protein damage scores of individuals using the gene sequence variation information; an individual drug safety score module which calculates individual drug safety scores of individuals and a population drug safety score module which calculates a population drug safety score; and a display unit which displays the values calculated by the calculation modules. In the present invention, a module may mean a functional or structural combination of hardware and software for driving the hardware for implementing the technical spirit of the present invention. For example, the module may be a predetermined code and a logical unit of a hardware resource by which the predetermined code is executed. It is obvious to those skilled in the art that the module does not necessarily mean physically connected codes or one kind of hardware.

Each “module” in the calculation unit 400 refers to a predetermined code and a logical unit of a hardware resource by which the predetermined code is executed for calculating each score on the basis of the gene sequence variation score, protein damage score, individual drug safety score, population drug safety score and information as grounds for calculation thereof with respect to a drug and a gene of analysis target according to the present invention, but does not necessarily mean physically connected codes or one kind of hardware.

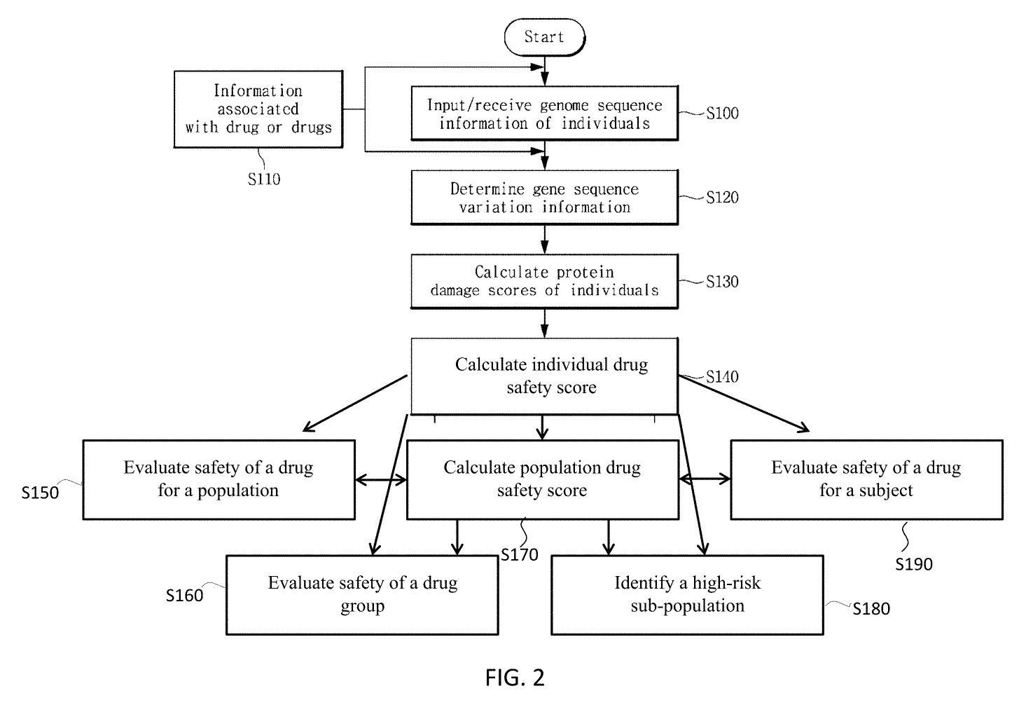

FIG. 2 illustrates each step of various methods for providing drug safety information and identifying a high-risk subpopulation using gene sequence variation information of a population according to an exemplary embodiment of the present invention. In some embodiments of the present invention, the method for providing drug safety information is performed by sequentially (1) receiving or being inputted with gene sequence variation information of individuals in a population (S100), (2) receiving or being inputted with information relevant to a particular drug or drugs (S110), (3) determining gene sequence variation information of the individuals (S120), (4) calculating protein damage scores of the individuals with respect to the particular drug or drugs (S130), and (5) calculating individual drug safety scores with respect to the particular drug or drugs (S140). The individual drug safety scores of individuals within a population can be used (1) to evaluate safety of a drug for a population (S150), (2) to evaluate safety of a drug group (S160), (3) to calculate a population drug safety score (S170), (4) to identify a high-risk sub-population (S180), or (5) to evaluate safety of a drug for a subject.

As one example, at step S100, the drug safety evaluation system might receive from a pharmaceutical company or research institution requesting a drug evaluation, or from a sequencing laboratory, the genome sequence information of multiple individuals of a population, and this data can be provided over a network. The information provided at S110 can include data about the drug being evaluated and possibly data about genes related to the pharmacodynamics or pharmacokinetics of the drug or the drugs. The genome sequence information of multiple individuals and the information associated with drug or drugs can be used at S120 to determine gene sequence variation information. The gene sequence variation information can be used at step S130 to calculate protein damage scores for each protein encoded by the gene associated with the drug. The protein damage scores can be used at step S140 to calculate individual drug safety score. When multiple genes are associated with a drug, the individual drug safety score can be calculated a mean of multiple protein damage scores, each corresponding to one of the multiple genes. The individual drug safety score is calculated for each of the multiple individuals within a given population, to generate a set of individual drug safety scores.

The set of individual drug safety scores can be used at S150 to evaluate safety of a drug for a population. In some embodiments, the evaluation is done by receiving a population drug safety score at S170. The population drug safety score can be calculated based on the set of individual drug safety scores at S170. In some embodiments, the population drug safety score is obtained by calculating a mean of the set of individual drug safety scores, or by measuring an area under the curve of the set of individual drug safety scores. The population drug safety score and the set of individual drug safety scores can be used to evaluate safety of the drug for a subject at S190. In some cases, drug safety to the subject can be determined by comparing an individual drug safety score of the subject with the population drug safety score, or by comparing the distribution of individual drug safety scores and the individual drug safety score of the subject.

In some embodiments, the set of individual drug safety scores or the population drug safety score can be used to evaluate safety of a drug group (S160). The drug groups may be determined based on known drug classification methods such as the Anatomical Therapeutic Chemical (ACT) Classification System of the WHO, drugs used for identical symptoms, drugs with similar chemical properties, drugs sharing pathways, drugs with identical absorption or excretion mechanisms, drugs with identical targets, etc., although not being limited thereto. Safety of a drug group can be calculated as a mean of population drug scores for the drugs within the drug group. In some embodiments, the set of individual drug safety scores and the population drug safety score can be used to identify a sub-population, which is likely to have adverse side-effect to a drug (S180). The high-risk sub-population can be identified by identifying individuals having an individual drug safety score below a threshold score.

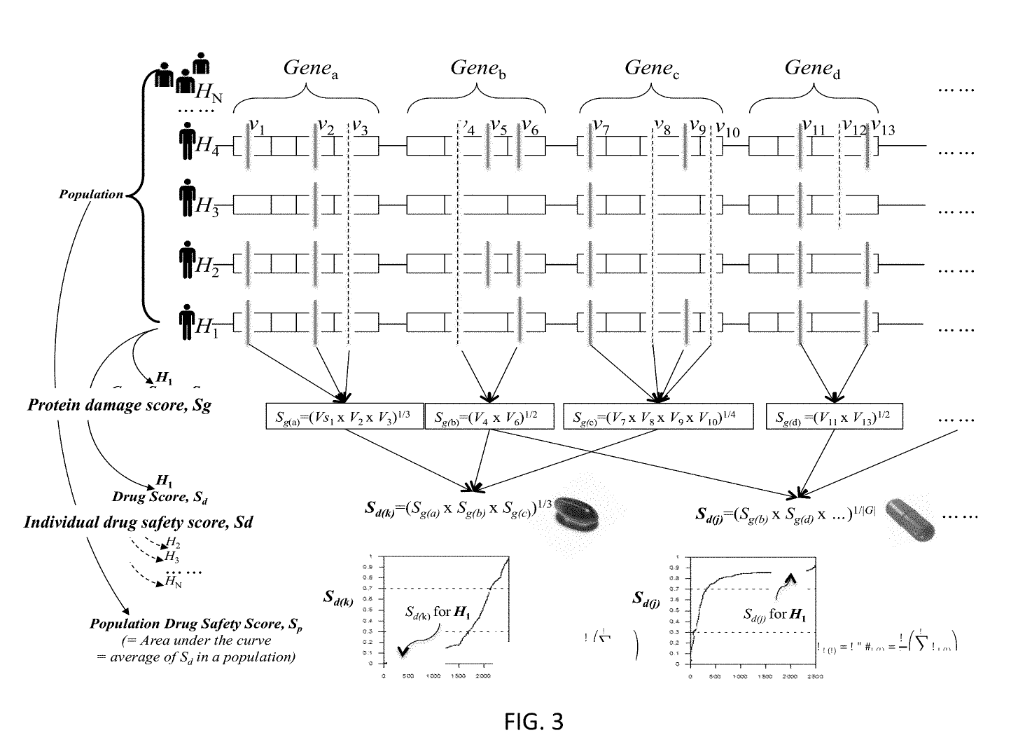

FIG. 3 schematically illustrates a method for calculating a population drug safety score and calculating a drug safety rank of an individual using gene sequence variation of individuals within a population according to an exemplary embodiment of the present invention. In some embodiments, the method comprises identifying gene sequence variation information (V1, V2, V3, . . . V12, V13) corresponding to genes (Gene a, b, c, and d) associated with pharmacodynamics and pharmacokinetics of a drug (d(k) or d(j)). This is performed across each of multiple individuals of a population or across all individuals of a population, such as individuals H₁, H₂, H₃, H₄, . . . Hn. Gene sequence variation information (V1, V2, V3, . . . V12, V13) is used to calculate protein damage scores (S_{g(a)}, S_{g(b)}, S_{g(c)}, and S_{g(d)}) for each individual, and for each of the genes a, b, c, and d. Protein damage scores for an individual are used to calculate an individual drug safety score (S_{d(k)} or S_{d(j)}) for each individual. Individual drug safety scores can be plotted as a distribution curve with individual drug safety score ranging from 0 to 1, as illustrated on the bottom of FIG. 3. A drug safety rank of an individual (e.g., H1) can be calculated by ranking the individual drug safety score of the individual from the lowest to the highest within the distribution curve. A population drug safety score (Sp) for the population can be calculated as an area under the distribution curve or as an average of individual drug safety scores (Sd) in the population, so this is a combined representation of the individual scores as a whole population score.

Hereinafter, the present invention will be described in more detail with reference to the following examples. The following examples are provided to explain the present invention in detail but do not limit the scope of the present invention.

6.3.2. Gene Sequence Variation Information

The present invention is based on the finding that it is possible to evaluate drug safety by analyzing gene sequence variation information of individuals within a population. The PCT/KR2014/007685A, incorporated by reference in its entirety, presents a method of inferring protein damage by analyzing gene sequence variation information of individual and calculating drug safety scores of individual based thereon. The methods of obtaining, calculating, and using gene sequence variation information disclosed in the application PCT/KR2014/007685A can be adopted in the methods disclosed herein.

In an aspect, the present invention relates to a method for calculating a drug safety score and identifying a high-risk subpopulation using gene sequence variation of a population, comprising: a step of determining one or more gene sequence variation information associated with the pharmacodynamics or pharmacokinetics of a particular drug or drugs from gene sequence information of individuals; a step of calculating protein damage scores of individuals using the gene sequence variation information; and a step of calculating individual drug safety scores of individuals and a population drug safety score of a population by correlating the protein damage scores of individuals with the interrelationship between the drug(s) and proteins.

The gene sequence variation information refers to information related to a gene sequence variation or polymorphism of individuals. In the present invention, the gene sequence variation or polymorphism occurs particularly in the exon region of a gene encoding proteins involved in the pharmacodynamics or pharmacokinetics of a drug or drugs, although not being limited thereto.

The term “sequence variation information” used in the present invention refers to information about substitution, addition or deletion of a nucleotide in a gene. The substitution, addition or deletion may result from many causes. For example, it may result from structural abnormality including breakage, deletion, duplication, inversion and/or translocation of a chromosome.

In another aspect, a polymorphism of a sequence refers to difference in a sequence present in a genome among individuals. In the polymorphism of a sequence, a single-nucleotide polymorphism (SNP) is the most frequent form. It refers to difference in one base of a sequence consisting of A, T, C and G. The sequence polymorphism including the SNP can be expressed as SNV (single nucleotide variation), STRP (short tandem repeat polymorphism) or a polyalleic variation including VNTR (variable number tandem repeat) and CNV (copy number variation).

In the method of the present invention, sequence variation or polymorphism information found in an individual genome is collected in association with a protein involved in the pharmacodynamics or pharmacokinetics of a particular drug or drugs. That is to say, the sequence variation information used in the present invention is variation information found particularly in the exon region of one or more genes involved in the pharmacodynamics or pharmacokinetics of a particular drug or drugs effective in treating a specific disease, for example, genes encoding a target protein relevant to the drug, an enzyme protein involved in drug metabolism, a transporter protein and a carrier protein, among the obtained genome sequence information of individuals, although not being limited thereto.

The genome sequence information of individuals used in the present invention may be determined by using a well-known sequencing method. Further, commercially available services such as those provided by Complete Genomics, BGI (Beijing Genome Institute), Knome, Macrogen, DNALink, etc. which provide commercialized services may be used, although not being limited thereto.

In the present invention, gene sequence variation information present in the genome sequence of individuals may be extracted by using various methods and may be acquired through sequence comparison analysis by using an algorithm such as ANNOVAR (Wang et al., Nucleic Acids Research, 2010; 38(16): e164), SVA (SequenceVariantAnalyzer) (Ge et al., Bioinformatics, 2011; 27(14): 1998-2000), BreakDancer (Chen et al., Nat Methods, 2009 September; 6(9): 677-81), etc., which compares a sequence to a reference group, for example, the genome sequence of HG19.

The gene sequence variation information may be obtained by various means. In some embodiments, gene sequence variation information is obtained by receiving/acquiring information through a computer system. In this aspect, the method of the present invention may further comprise a step of receiving the gene sequence variation information through a computer system. In some embodiments, gene sequence variation information is obtained from a storage device or database. In some embodiments, gene sequence variation information is obtained by analyzing genome sequences.

The computer system used in the present invention may include or access one or more databases containing information about the gene involved in the pharmacodynamics or pharmacokinetics of a particular drug or drugs, for example, a gene encoding a target protein relevant to the drug, an enzyme protein involved in drug metabolism, a transporter protein, a carrier protein, etc. These databases may include a public or non-public database or a knowledge base, which provides information about gene/protein/drug-protein interaction, etc., including, e.g., DrugBank (http://drugbank.ca/), KEGG DRUG (http://www.genome.jp/kegg/drug/), PharmGKB (http://www.pharmgkb.org/), etc., although not being limited thereto.

In the present invention, the particular drug or drugs may be information input by a user, information input from a prescription or information input from a database containing information about a drug effective in treating a specific disease. The prescription may include an electronic prescription, although is limited thereto.

The term “gene sequence variation score” used in the present invention refers to a numerical score of a degree of the individual gene sequence variation, when the gene sequence variation is found in the exon region of the gene encoding the protein, that causes an amino acid sequence variation (substitution, addition or deletion) of a protein encoded by a gene or a variation in transcription regulation and thus causes a significant change in the protein expression. The gene sequence variation score can be calculated considering a degree of evolutionary conservation of amino acids in a genome sequence, a degree of an effect of a physical characteristic of modified amino acids on the structure or function of the corresponding protein, etc.

In an exemplary embodiment of the present invention, the SIFT (Sorting Intolerant From Tolerant) algorithm is used to calculate an individual gene sequence variation score. In the case of the SIFT algorithm, gene sequence variation is input in the form of, e.g., a VCF (Variant Call Format) file and a degree of damage caused by each gene sequence variation to the corresponding gene is scored. In the case of the SIFT algorithm, as a calculated score is closer to 0, it is considered that a protein encoded by a corresponding gene is severely damaged and thus its function is damaged, and as the calculated score is closer to 1, it is considered that the protein encoded by the corresponding gene maintains its normal function.

In the case of another algorithm PolyPhen-2, the higher a calculated score is, it is considered that the more damaged is a function of a protein encoded by a corresponding gene.

Recently, a study suggesting the Condel algorithm by comparing and combining SIFT, PolyPhen-2, MAPP, Logre and Mutation Assessor was reported (Gonzalez-Perez, A. & Lopez-Bigas, N., Improving the assessment of the outcome of nonsynonymous SNVs with a consensus deleteriousness score, Condel. The American Journal of Human Genetics 2011; 88: 440-449). In this study, the above-described five algorithms are compared by using HumVar and HumDiv (Adzhubei, I A et al., A method and server for predicting damaging missense mutations. Nature Methods, 2010; 7(4): 248-249) as a set of known data relating to gene sequence variations damaging a protein and gene sequence variations with less effect. As a result, 97.9% of gene sequence variations damaging a protein and 97.3% of gene sequence variations with less effect of HumVar were identically detected by at least three of the above-described five algorithms, and 99.7% of gene sequence variations damaging a protein and 98.8% of gene sequence variations with less effect of HumDiv were identically detected by at least three of the above-described five algorithms. Further, as a result of drawing an ROC (Receiver Operating Curve) showing the accuracy of calculation results of the five algorithms and a combination of the algorithms utilizing HumDiv and HumVar, it was confirmed that an AUC (Area Under the Receiver Operating Curve) consistency is considerably high (69-88.2%). That is to say, the above-described algorithms are different in calculation method but the calculated gene sequence variation scores are significantly correlated to each other. Therefore, it is included in the scope of the present invention to apply a gene sequence variation score calculated by any of the above-described algorithms or a method employing any of the algorithms to the steps of calculating an individual protein damage score and an individual drug safety score according to the present invention.

6.3.3. Protein Damage Score

The gene sequence variation score can be used for calculating the protein damage scores of individuals. For example, the protein damage score can be calculated from the gene sequence variation information by using an algorithm such as SIFT (Sorting Intolerant From Tolerant, Pauline C et al., Genome Res. 2001 May; 11(5): 863-874; Pauline C et al., Genome Res. 2002 March; 12(3): 436-446; Jing Hul et al., Genome Biol. 2012; 13(2): R9), PolyPhen, PolyPhen-2 (Polymorphism Phenotyping, Ramensky V et al., Nucleic Acids Res. 2002 Sep. 1; 30(17): 3894-3900); Adzhubei et al., Nat Methods 7(4): 248-249 (2010)), MAPP (Eric A. et al., Multivariate Analysis of Protein Polymorphism, Genome Research 2005; 15: 978-986), Logre (Log R Pfam E-value, Clifford R. J. et al., Bioinformatics 2004; 20: 006-1014), Mutation Assessor (Reva B et al., Genome Biol. 2007; 8: R232, http://mutatioassessor.org/), Condel (Gonzalez-Perez A et al., The American Journal of Human Genetics 2011; 88: 440-449, http://bg.upfedu/fannsdb/), GERP (Cooper et al., Genomic Evolutionary Rate Profiling, Genome Res. 2005; 15; 901-913, http://mendel.standford.edu/SidowLab/downloads/gerp/), CADD (Combined Annotation-Dependent Depletion, http://cadd.gs.washington.edu/), MutationTester, MutationTester2 (Schwarz et al., MutationTester2: mutation prediction for the deep-sequencing age. Nature Methods 2014; 11: 361-362, http://www.mutationtester.org/), PROVEAN (Choi et al., PLoS One 2012; 7(10): e46688), PMut (Ferrer-Costa et al., Proteins 2004; 57(4): 811-819, http://mmb.pcb.ub.es/PMut/), CEO (Combinatorial Entropy Optimization, Reva et al., Genome Biol. 2007; 8(11): R232), SNPeffect (Reumers et al., Bioinformatics 2006; 22(17): 2183-2185, http://snpeffect.vib.be), FATHMM (Shihab et al., Functional Analysis through Hidden Markov Models, Hum Mutat 2013; 34: 57-65, http://fathmm.biocompute.org.uk/), etc., although not being limited thereto.

The above-described algorithms are configured to identify how much each gene sequence variation has an effect on a protein function or whether or not there are any other effects. These algorithms have common aspects in that they are basically configured to consider an amino acid sequence of a protein encoded by a corresponding gene and relevant effects caused by an individual gene sequence variation and thereby to determine an effect on a structure and/or function of the corresponding protein.

The term “protein damage score” used in the present invention refers to a score calculated based on gene sequence variation scores when two or more significant sequence variations are found in a gene encoding a single protein so that the single protein has two or more gene sequence variation scores. If there is a single significant sequence variation in the gene region encoding the protein, a gene sequence variation score is identical to a protein damage score. If there are two or more gene sequence variations encoding the protein, a protein damage score is calculated as a mean of gene sequence variation scores calculated for the respective variations. Such a mean can be calculated, for example, as a geometric mean, an arithmetic mean, a harmonic mean, an arithmetic-geometric mean, an arithmetic-harmonic mean, a geometric-harmonic mean, a Pythagorean mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication or a weighted multiplication, or by a functional operation of the calculated values, although not being limited thereto.

In an exemplary embodiment of the present invention, the protein damage score is calculated by the following Equation 1. The following Equation 1 can be modified in various ways, and, thus, the present invention is not limited thereto.

$${S}_{g} ( {v}_{1 , … ,} {v}_{n} ) = {( \frac{1}{n} {\sum }_{i = 1}^{n} {v}_{i}^{p} )}^{\frac{1}{p}} \qquad [ Equation 1 ]$$

In Equation 1, S_{g} is a protein damage score of a protein encoded by a gene g, n is the number of target sequence variations for analysis among sequence variations of the gene g, vᵢ is a gene sequence variation score of an i-th gene sequence variation, and p is a real number other than 0. In Equation 1, when a value of the p is 1, the protein damage score becomes an arithmetic mean, if the value of the p is −1, the protein damage score becomes a harmonic mean, and if the value of the p is close to the limit 0, the protein damage score becomes a geometric mean.

In another exemplary embodiment of the present invention, the protein damage score is calculated by the following Equation 2.

$${S}_{g} ( {υ}_{1 , … ,} {υ}_{n} ) = {( {\prod }_{i = 1}^{n} {υ}_{i}^{{w}_{i}} )}^{1 / {\sum }_{i = {1}^{{w}_{i}}}^{n}} \qquad [ Equation 2 ]$$

In Equation 2, S_{g} is a protein damage score of a protein encoded by a gene g, n is the number of target sequence variations for analysis among sequence variations of the gene g, vᵢ is a gene sequence variation score of an i-th gene sequence variation, and wᵢ is a weighting assigned to the vᵢ. If all weightings wᵢ have the same value, the protein damage score S_{g} becomes a geometric mean of the gene sequence variation scores vᵢ. The weighting may be assigned considering a class of the corresponding protein, pharmacodynamic or pharmacokinetic classification of the corresponding protein, pharmacokinetic parameters of the enzyme protein of a corresponding drug, a population group, or a race distribution.

6.3.4. Individual Drug Safety Score

According to the method of the present invention, an individual drug safety score is calculated by associating the above-described protein damage score with a drug-protein relation.

In one embodiment, if two or more proteins involved in the pharmacodynamics or pharmacokinetics of a particular drug or drugs are damaged, a drug safety score is calculated as a mean of the protein damage scores. Such a mean can be calculated, for example, as a geometric mean, an arithmetic mean, a harmonic mean, an arithmetic-geometric mean, an arithmetic-harmonic mean, a geometric-harmonic mean, a Pythagorean mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication or a weighted multiplication, or by a functional operation of the calculated values, although not being limited thereto.

The individual drug safety score may be calculated by adjusting weightings of a target protein involved in the pharmacodynamics or pharmacokinetics of the corresponding drug, an enzyme protein involved in drug metabolism, a transporter protein or a carrier protein in consideration of pharmacological characteristics, and the weighting may be assigned considering pharmacokinetic parameters of the enzyme protein of a corresponding drug, a population group, a race distribution, or the like. Further, although not directly interacting with the corresponding drug, proteins interacting with a precursor of the corresponding drug and metabolic products of the corresponding drug, for example, proteins involved in a pharmacological pathway, may be considered, and protein damage scores thereof may be combined to calculate the individual drug safety score. Further, protein damage scores of proteins significantly interacting with the proteins involved in the pharmacodynamics or pharmacokinetics of the corresponding drug may also be considered and combined to calculate the individual drug safety score. Information about proteins involved in a pharmacological pathway of the corresponding drug, which significantly interact with the proteins in the pathway or are involved in a signal transduction pathway thereof, can be searched in publicly known biological databases such as PharmGKB (Whirl-Carrillo et al., Clinical Pharmacology & Therapeutics 2012; 92(4): 414-4171), the MIPS Mammalian Protein-Protein Interaction Database (Pagel et al., Bioinformatics 2005; 21(6): 832-834), BIND (Bader et al., Biomolecular Interaction Network Database, Nucleic Acids Res. 2003 Jan. 1; 31(1): 248-50), Reactome (Joshi-Tope et al., Nucleic Acids Res. 2005 Jan. 1; 33 (Database issue): D428-32), etc.

In an exemplary embodiment of the present invention, the individual drug safety score is calculated by the following Equation 3. The following Equation 3 can be modified in various ways, and, thus, the present invention is not limited thereto.

$${S}_{d} ( {g}_{1 , … ,} {g}_{n} ) = {( \frac{1}{n} {\sum }_{i = 1}^{n} {g}_{i}^{p} )}^{\frac{1}{p}} \qquad [ Equation 3 ]$$

In Equation 3, S_{d} is an individual drug safety score of a drug d, n is the number of proteins directly involved in the pharmacodynamics or pharmacokinetics of the drug d or interacting with a precursor of the corresponding drug or metabolic products of the corresponding drug, for example, proteins encoded by one or more genes selected from a gene group involved in a pharmacological pathway, gᵢ is a protein damage score of a protein directly involved in the pharmacodynamics or pharmacokinetics of the drug d or interacting with a precursor of the corresponding drug or metabolic products of the corresponding drug, for example, a protein encoded by one or more genes selected from a gene group involved in a pharmacological pathway, and p is a real number other than 0. In Equation 3, when a value of the p is 1, the drug safety score becomes an arithmetic mean, if the value of the p is −1, the drug safety score is becomes harmonic mean, and if the value of the p is close to the limit 0, the individual drug safety score becomes a geometric mean.

In yet another exemplary embodiment of the present invention, the individual drug safety score is calculated by the following Equation 4.

$${S}_{d} ( {g}_{1 , … ,} {g}_{n} ) = {( {\prod }_{i = 1}^{n} {g}_{i}^{{w}_{i}} )}^{1 / {\sum }_{i = {1}^{{w}_{i}}}^{n}} \qquad [ Equation 4 ]$$

In Equation 4, S_{d} is an individual drug safety score of a drug d, n is the number of proteins directly involved in the pharmacodynamics or pharmacokinetics of the drug d or interacting with a precursor of the corresponding drug or metabolic products of the corresponding drug, for example, proteins encoded by one or more genes selected from a gene group involved in a pharmacological pathway, gᵢ is a protein damage score of a protein directly involved in the pharmacodynamics or pharmacokinetics of the drug d or interacting with a precursor of the corresponding drug or metabolic products of the corresponding drug, for example, a protein encoded by one or more genes selected from a gene group involved in a pharmacological pathway, and wᵢ is a weighting assigned to the gᵢ. If all weightings wᵢ have the same value, the individual drug safety score S_{d} becomes a geometric mean of the protein damage scores gᵢ. The weighting may be assigned considering the kind of the protein, the pharmacodynamic or pharmacokinetic classification of the protein, the pharmacokinetic parameters of the enzyme protein of the corresponding drug, a population group or a race distribution.

In the case of a geometric mean calculation method used in an exemplary embodiment of the present invention, weightings are equally assigned regardless of the characteristic of a drug-protein relation. However, it is possible to calculate a drug safety score by assigning weightings considering each characteristic of a drug-protein relation as described in yet another exemplary embodiment. For example, different scores may be assigned to a target protein of a drug and a transporter protein related to the drug. Further, it is possible to calculate an individual drug safety score by assigning the pharmacokinetic parameters Kₘ, Vₘₐₓ, and K_{cat}/Kₘ as weightings to the enzyme protein of a corresponding drug. Furthermore, for example, since a target protein is regarded more important than a transporter protein in terms of pharmacological action, it may be assigned a higher weighting, or a transporter protein or a carrier protein may be assigned high weightings with respect to a drug whose effectiveness is sensitive to a concentration, but the present invention is not limited thereto. The weighting may be minutely adjusted according to the characteristics of a relation between a drug and a protein related to the drug and the characteristics of an interaction between the drug and the protein. A sophisticated algorithm configured to assign a weighting considering the characteristic of an interaction between a drug and a protein can be used. For example, a target protein and a transporter protein may be assigned 2 points and 1 point, respectively.

In the above description, only the protein directly interacting with a drug has been exemplified. However, as in an exemplary embodiment of the present invention, the predictive ability of the above equations can be improved by using information about the protein interacting with a precursor of the corresponding drug or metabolic products of the corresponding drug, the protein significantly interacting with proteins involved in the pharmacodynamics or pharmacokinetics of the corresponding drug, and the protein involved in a signal transduction pathway thereof. That is to say, by using information about a protein-protein interaction network or pharmacological pathway, it is possible to use information about various proteins relevant thereto. That is to say, even if a significant variation is not found in the protein directly interacting with the drug and thus there is no protein damage score calculated with respect to the protein or there is no damage (for example, 1.0 point when the SIFT algorithm is applied), a mean (for example, a geometric mean) of protein damage scores of proteins interacting with the protein or involved in the same signal transduction pathway of the protein may be used as a protein damage score of the protein so as to be used for calculating an individual drug safety score.

The individual drug safety score can be calculated with respect to all the drugs from which information about one or more associated proteins can be acquired or some drugs selected from the drugs. Further, the individual drug safety score can be converted into a rank.

6.3.5. Population Drug Safety Score

In some embodiments of the present invention, a population drug safety score is calculated by using individual drug safety scores.

The term “population drug safety score” used in the present invention refers to a mean of individual drug safety scores of individuals belonging to a particular population for a drug. The population drug safety score can be obtained by calculating the area under the curve (AUC) of a individual drug safety score distribution curve, a curve obtained by plotting the drug safety scores of individuals belonging to the population from lower to higher scores, and dividing the AUC by the number of the individuals constituting the population. This is called a standardized area under the curve (S-AUC). When all the drug safety scores in a population are 1, i.e., when there is no variation in drug-related genes which cause functional abnormality of proteins, the area under the curve is equal to the number of the individuals constituting the population. Similarly, the value obtained by dividing the area upper the individual drug safety score distribution curve by the number of the individuals constituting the population is called a standardized area upper the curve (S-AUPC) and it can be used as the population drug safety score. 1-(S-AUPC), which is equal to S-AUC, can also be used as the population drug safety score.

The population drug safety score may be calculated for individual drugs or drug groups considering the characteristics of the drugs. The drug groups may be determined based on known drug classification methods such as the Anatomical Therapeutic Chemical (ACT) Classification System of the WHO, drugs used for identical symptoms, drugs with similar chemical properties, drugs sharing pathways, drugs with identical absorption or excretion mechanisms, drugs with identical targets, etc., although not being limited thereto.

In an exemplary embodiment of the present invention, the population drug safety score is calculated by Equation 5. However, Equation 5 can be modified variously and the present invention is not limited thereto.

$${S}_{p} ( d 1 , … , dn ) = \frac{1}{N} ( {\sum }_{H = 1}^{N} {S}_{d} ) \qquad [ Equation 5 ]$$

In Equation 5, S_{P} is a population drug safety score calculated as a mean of individual drug safety scores of individuals within a population, N or n is the number of individuals for which the individual drug safety score d are calculated through individual genetic variation analysis and S_{d} is the an individual drug safety score of a subject individual. The population may be defined variously based on sex, age, race, disease group, drug medication group, etc., although not being limited thereto. The population drug safety score may be different among different populations.

$${S}_{p} ( d 1 , … , dn ) = \frac{1}{N} ( {AUC}_{d} ) = 1 - \frac{1}{N} ( {AUPC}_{d} ) \qquad [ Equation 6 ]$$

In Equation 6, S_{P} is a population drug safety score calculated as a mean of individual drug safety scores d₁₋ₙ of individuals within a population, AUC_{d} is an area under the individual drug safety score distribution curve for the population, AUPC_{d} is an area upper the individual drug safety score distribution curve for the population and N is the number of individuals for which the individual drug safety scores d are calculated through individual genetic variation analysis. The value obtained by dividing AUC by the number of the individuals belonging to the population is a standardized area under the curve. The value obtained by dividing AUPC by the number of the individuals belonging to the population is a standardized area upper the curve. The population may be defined variously based on sex, age, race, disease group, drug medication group, etc., although not being limited thereto. The population drug safety score may be different among different populations.

The term “individual drug safety score distribution curve” or “distribution curve of individual drug safety scores” used in the present invention refers to a plot of the distribution of individual drug safety scores of individuals within a particular population. It includes a line graph obtained by plotting the individual drug safety scores from lower to higher scores, a density curve plotted using a density estimation function, a histogram, etc., although not being limited thereto. Further, the population herein may be defined variously based on sex, age, race, disease group, drug medication group, etc., although not being limited thereto. The population drug safety score may be different with respect to different populations and drugs.

6.3.6. Applications

6.3.6.1. Identification of a High-Risk Subpopulation

In an exemplary embodiment of the present invention, the drug safety threshold score for identifying a high-risk subpopulation is calculated by Equation 7. However, Equation 7 can be modified and the present invention is not limited thereto.

$$T = μ - κ \sqrt{\frac{1}{n} {\sum }_{i = 1}^{n} {( {d}_{i} - μ )}^{2}} \qquad [ Equation 7 ]$$

In Equation 7, T is a drug safety threshold score calculated based on S-AUC from the individual drug safety score distribution curve, or an arithmetic mean of individual drug safety scores d of a population. T is a rational number satisfying 0<T<1. N is the number of individuals for which the individual drug safety scores d are calculated through individual genetic variation analysis, dᵢ is an individual drug safety score of i-th individual and μ is a population drug safety score calculated as an arithmetic mean or a standardized area under the individual drug safety score distribution curve, and κ is an non-zero rational number. When κ is 1, T becomes a score corresponding to the population drug safety score μ subtracted by standard deviation of the individual drug safety scores. When κ is 2, it becomes a score corresponding to the population drug safety score μ subtracted by 2 times of standard deviations of the individual drug safety scores. κ may be varied depending on the distribution of individual drug safety scores within the population. The population may be defined variously based on sex, age, race, disease group, drug medication group, etc., although not being limited thereto. The drug safety threshold score may be different for different populations and drugs.

The term “high-risk subpopulation” used in the present invention refers to a set of individuals having drug safety scores equal to or lower than the drug safety threshold score. It is a subpopulation having many variations causing damage of proteins associated with the pharmacodynamics or pharmacokinetics of the corresponding drug and which is vulnerable to the drug. The drug safety threshold score may be determined based on the pattern of the individual drug safety score distribution curve. That is to say, when there is a subpopulation which forms an island with a remarkably low score distribution in the individual drug safety score distribution curve of the drug, the drug safety threshold score may be calculated as an individual drug safety score defining the island.

R={x|x with d<T} [Equation 8]

In Equation 8, R is the ratio or fraction of a high-risk subpopulation with a score lower than the drug safety threshold score in a population, x is an individual with an individual drug safety score (d) lower than the drug safety threshold score. The population may be defined variously based on sex, age, race, disease group, drug medication group, etc., although not being limited thereto. The drug safety threshold score may be different for different populations and drugs.

In another exemplary embodiment of the present invention, the threshold score can be estimated through analysis of drug safety scores corresponding to drugs which are withdrawn from the market or whose use has been restricted.

R={x|x with d≦T_{w}} [Equation 9]

In Equation 9, R is the ratio or fraction of a high-risk subpopulation with a score lower than the drug safety threshold score in a population, x is an individual with an individual drug safety score lower than the drug safety threshold score and d is an individual drug safety score. In some embodiments, T_{w} is 0.3 as calculated based on drugs which are withdrawn from the market or whose use has been restricted. The population may be defined variously based on sex, age, race, disease group, drug medication group, etc., although not being limited thereto. The drug safety threshold score may be different for different populations and drugs and is not limited to 0.3.

Once a high-risk subpopulation is identified, the result can be used by a drug maker, a company running clinical studies, or other pharmaceutical companies in developing a drug, designing clinical studies or selling the drug targeted to a specific population. The result can be also used by physicians when they decide whether to prescribe a certain drug or not. The result can be also used by patients when they decide whether to use a certain drug or not.

6.3.6.2. Evaluation of Safety of Drug for a Subject

In some embodiments, the individual drug safety scores distribution curve can be used to evaluate safety of drug for a subject. For example, an individual drug safety score of the subject can be compared with the individual drug safety scores of multiple individuals within the population or the distribution curve of the scores. If the subject has an individual drug safety score lower than the threshold score described above, or lower than a majority of the individuals in the population, the subject is more likely to have variations in the genes associated with the pharamodynamics and pharmacokinetics of the drug and is more likely to show an undesired side-effect to the drug. Similar analysis can be performed for a number of drugs within a drug group, in order to identify a safest drug to use within the drug group.

Results from the analysis can be provided to the subject or to a physician for the subject. The physician may rely on the results to prescribe the drug, for example, by adjusting a dosage of the drug. Thus, the method of the present invention may be performed for the purpose of preventing side effects of a drug, although not being limited thereto.

### 6.4. Examples

The following examples are provided by way of illustration not limitation.

### 6.4.1. Example 1

Validity of the methods disclosed in the present invention is demonstrated through analysis of sequence variation information found in gene involved in pharmacodynamics or pharmacokinetics of drug withdrawn from market.

Any drug approved by the FDA and sold in the market can be ordered to be withdrawn from the market according to a result of a post-market surveillance (PMS) while being widely used. Such withdrawal of a drug from the market is a medically critical issue. Even a drug approved after the whole process of a strict clinical trial may cause unpredicted side effects in an actual application step with enormous sacrifices of life and economic losses and thus may be withdrawn. Differences in individual responses which cannot be found even with a large-scale clinical trial are regarded as one of the causes for withdrawal of a drug from the market. The method for identifying a high-risk subpopulation according to the present invention provides a method for testing the drug with the high-risk subpopulation and the low-risk subpopulation separately, approving the drug target to a specific subpopulation, and prescribing the drug or adjusting dosages of the drug depending on whether or not a subject belongs to a high-risk group or a low-risk group.

For the validation, gene sequence variation information of 2504 individuals was analyzed for 1041 drugs including drugs withdrawn from the market or restricted to use. In order to construct a comprehensive list of drugs withdrawn from the market, the list of withdrawn drugs from the European Medicines Agency (EMA) and “Consolidated List of Products Whose Consumption and/or Sale Have Been Banned, Withdrawn, Severely Restricted, or Not Approved by Governments: Pharmaceuticals” Versions 8, 10, 12 and 14 as the most comprehensive data about the drugs withdrawn from the worldwide market issued by the U.N. were reviewed overall in addition to the already included list of drugs withdrawn from the market from the DrugBank database. Finally, a list of 578 drugs withdrawn from at least one country was constructed, and it was confirmed that 154 drugs of them were included in the above-described 1041 drugs. Further, as the drugs which were not withdrawn from the market but are severely restricted to use, 260 drugs including 137 drugs from the Beers Criteria for Potentially Inappropriate Medication Use in Older Adults published since 2003 by the American Geriatrics Society and 148 drugs which were ordered by the US FDA to mark pharmacogenetics information on the drug label were included as precautionary drugs. Analysis was conducted for 165 drugs among the 260 drugs, which are included in the 1041 drugs. A population drug safety score of each drug was obtained by calculating gene sequence variation scores using the SIFT algorithm on the basis of genome sequence variations of the 2504 persons and acquiring an arithmetic mean of 2504 individual drug safety scores calculated from the gene sequence variation scores. As a result, the population drug safety scores of the withdrawn group, the restricted group (the drugs from the Beers Criteria and the FDA pharmacogenetics database) and the other group were 0.558±0.17, 0.549±0.15, 0.542±0.15 and 0.635±0.19, respectively, and as a result of an one-way analysis of variance, the difference thereof was significant (F=17.54, p<0.001). Further, as a result of a post-Tukey analysis, the p-value between the withdrawn drug and the other drug and the p-value between the restricted drug and the other drug showed statistical significance, with p<0.001. No significant difference was found between the withdrawn drug and the restricted drug (p-value for the withdrawn drugs vs. the FDA pharmacogenetics drugs=0.889; p-value for the withdrawn drugs vs. the Beers Criteria drugs=0.978; p-value for the FDA pharmacogenetics drugs vs. the Beers Criteria drugs=0.994). That is to say, it can be seen that in the studied population group, drugs having lower population drug safety scores are significantly more likely to be withdrawn from the market or restricted to use.

### 6.4.2. Example 2

The individual drug safety scores show a wide distribution from the minimum score 0 to the maximum score 1 depending on the variety of the individual genetic variation found in drug-related genes. If there is no functional variation in the genes associated with the pharmacodynamics or pharmacokinetics of a drug in a particular population group, all the drugs safety scores will be 1. Hence, the area under the individual drug safety score distribution curve will be 1, and the effect of the drug will be achieved as expected.

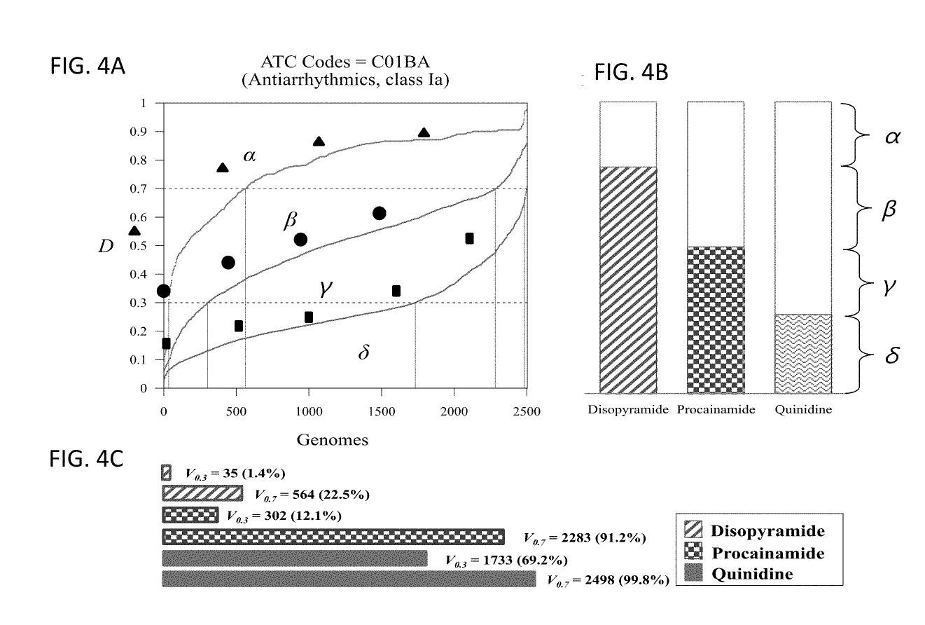

FIG. 4A-C presents graphs demonstrating methods for evaluating drug safety using individual drug safety scores calculated as described above. FIG. 4A shows three distribution curves representing individual drug safety scores from 2504 individuals (provided by the 1000 Genomes Project, Phase III), each corresponding to one of three drugs—disopyramide, procainamide and quinidine, which belong to C01BA antiarrythmics according to the ATC classification system. The drugs have been withdrawn from the market according to DrugBank, the UN and the EMA. The top curve (with triangles) corresponds to disopyramids, the middle curve (with circles) corresponds to procainamide, and the bottom curve (with rectangles) corresponds to quinidine. The distribution curves show that individual drug safety scores corresponding to each drug have different shapes and patterns. FIG. 4B provides bar graphs, each representing an area under the curve (AUC) for each drug. As shown on the right side of the graph, AUC for disopyramide is measured as 1−α, AUC for procainamide is measured as 1−(α+β), and AUC for quinidine is measured as 1−(α+β+Υ). FIG. 4C provides three bar graphs representing individual drug safety scores corresponding to the bottom 30% or 70% in the distribution of individual drug safety scores for each drug. The top two bars are for disopyramide, the middle two bars are for procainamide, and the bottom two bars are for quinidine.

### 6.4.3. Example 3

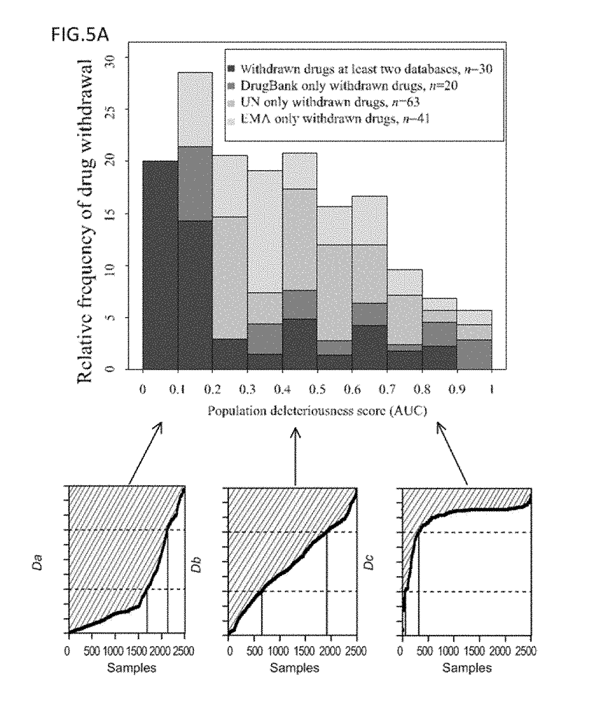

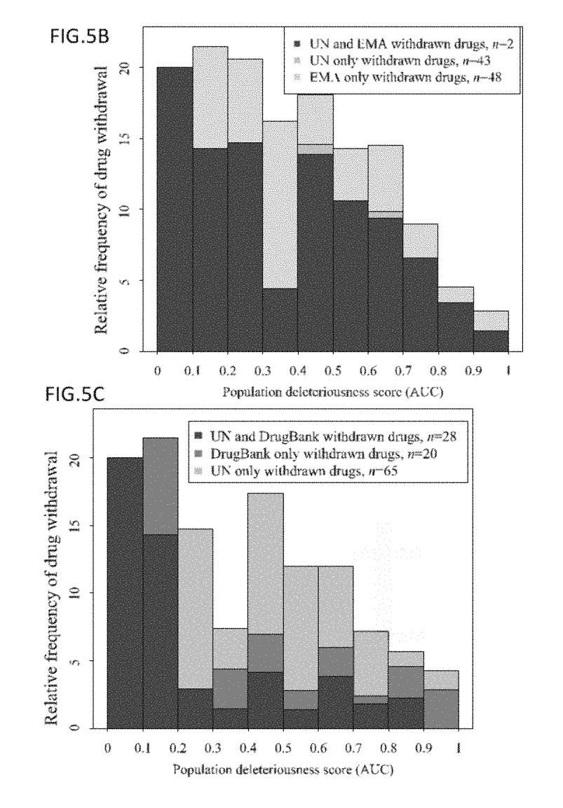

Population drug safety scores of various drugs were calculated as the area under the curve (AUC) of 2504 individual drug safety scores and visualized in the relative frequency histograms of FIGS. 5A-I. Each drug is respectively allotted to one of the 10 score sections between 0 and 1 based on its population drug score (x-axis, also called as “Population deleteriousness score”), and then, withdrawal rates of the drugs corresponding to the respective score sections are presented on the y-axis (“relative frequency of drug withdrawal”). The withdrawal rates are calculated based on information available from various databases, including DrugBank, the UN and the EMA database.

FIG. 5A provides drug withdrawal rates based on at least two databases (n=30, the darkest), based on DrugBank (n=20, the second darkest), based on UN (n=63, the third darkest), and based on EMA (n=41, the lightest). FIG. 5B provides drug withdrawal rates based on UN and EMA (n=2, the darkest), based on UN only (n=43, the second darkest), and based on EMA only (n=48, the lightest). FIG. 5C provides drug withdrawal rates based on UN and DrugBank (n=28, the darkest), based on DrugBank only (n=20, the second darkest), and based on UN only (n=65, the lightest). FIG. 5D provides drug withdrawal rates based on EMA only (n=43, the darker), and based on DrugBank only (n=48, the lighter). FIG. 5E provides drug withdrawal rates based on UN (n=93). FIG. 5F provides drug withdrawal rates based on EMA (n=43). FIG. 5G provides drug withdrawal rates based on DrugBank (n=48). FIG. 5H provides drug withdrawal rates based on FDA pharmacogenomics drugs (n=96). FIG. 5I provides drug withdrawal rates based on Beers criteria (n=90).

FIG. 5A further provides three distribution curves of individual drug safety scores for three different drugs—the first drug with a population drug safety score between 0 and 0.1, the second drug with a population drug safety score between 0.4 and 0.5, and the third drug with a population drug safety score between 0.8 and 0.9.

This analysis shows that drugs having a lower population drug safety score are more likely to be withdrawn from the market or restricted to use. In particular, drugs with a population drug safety score below 0.3 were significantly more likely to be withdrawn from the market or restricted to use.

### 6.4.4. Example 4

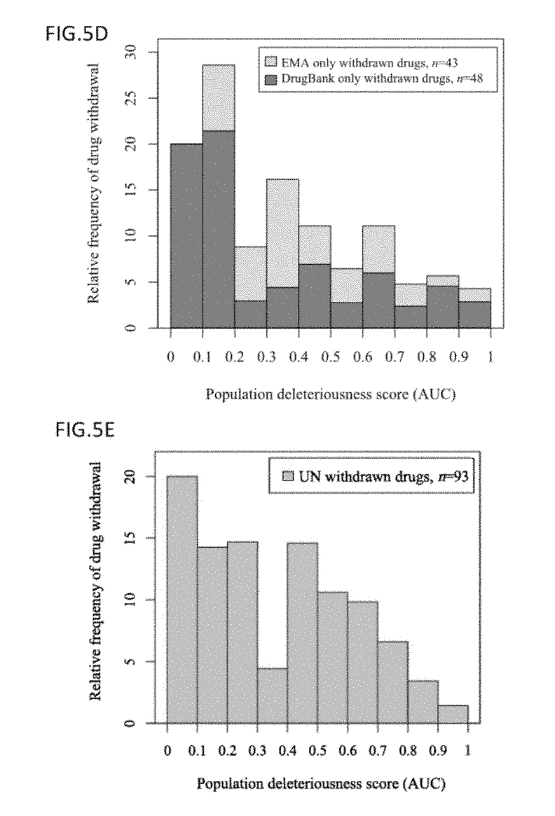

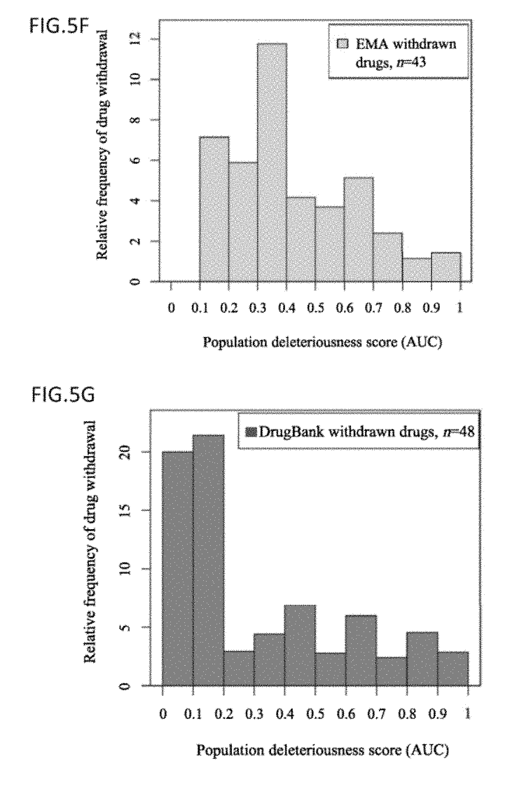

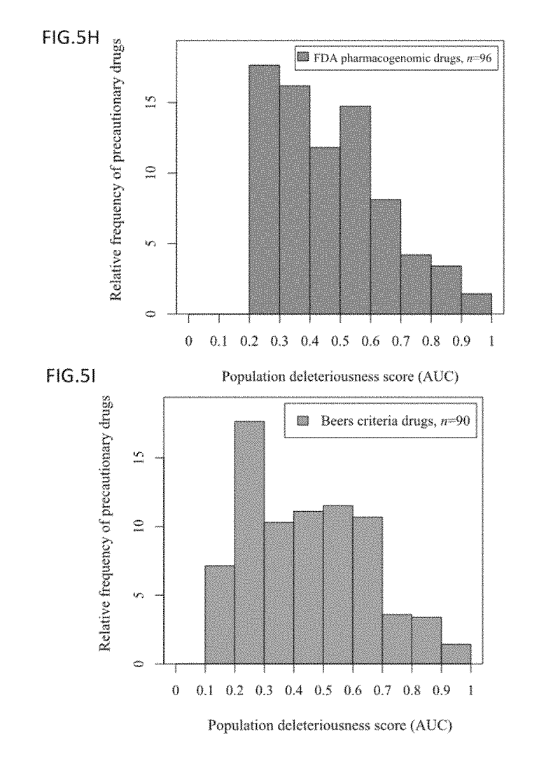

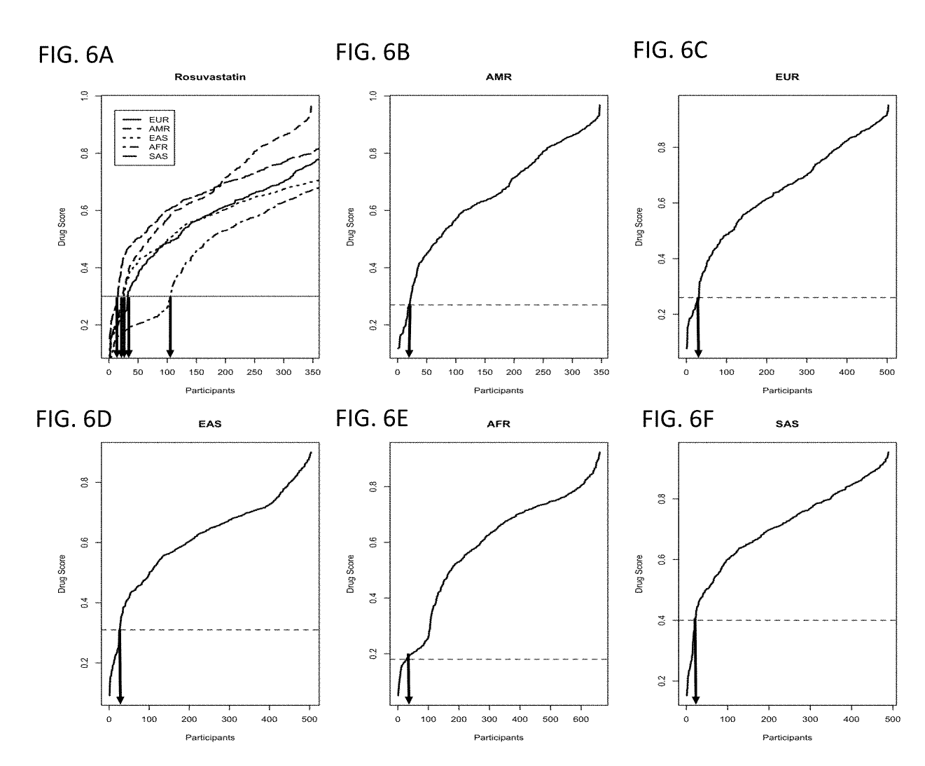

Applicants further demonstrated that distribution of individual drug safety scores have different patterns for different population as shown in FIGS. 6A-F. FIGS. 6A-F provides graphs, each representing a distribution curve of individual drug safe scores for Rosuvastatin. Each graph corresponds to one of five race groups—FIG. 6B for American (AMR), FIG. 6C for European (EUR), FIG. 6D for East Asian (EAS), FIG. 6E for African (AFR), FIG. 6F for South Asian (SAS), and FIG. 6A for a combination of all five race groups. The arrows in FIG. 6A represents rankings of the individuals having 0.3 as an individual drug safety score within the corresponding population. The arrows in FIGS. 6B-F represent an individual having the same ranking (30) of the individual drug safety score in the corresponding race group. This analysis demonstrates that each population group has different genetic variations in genes related to pharmacodynamics and pharmacokinetics of Rosuvastatin, and the difference can be identified by the methods of the present invention.

### 6.4.5. Example 5

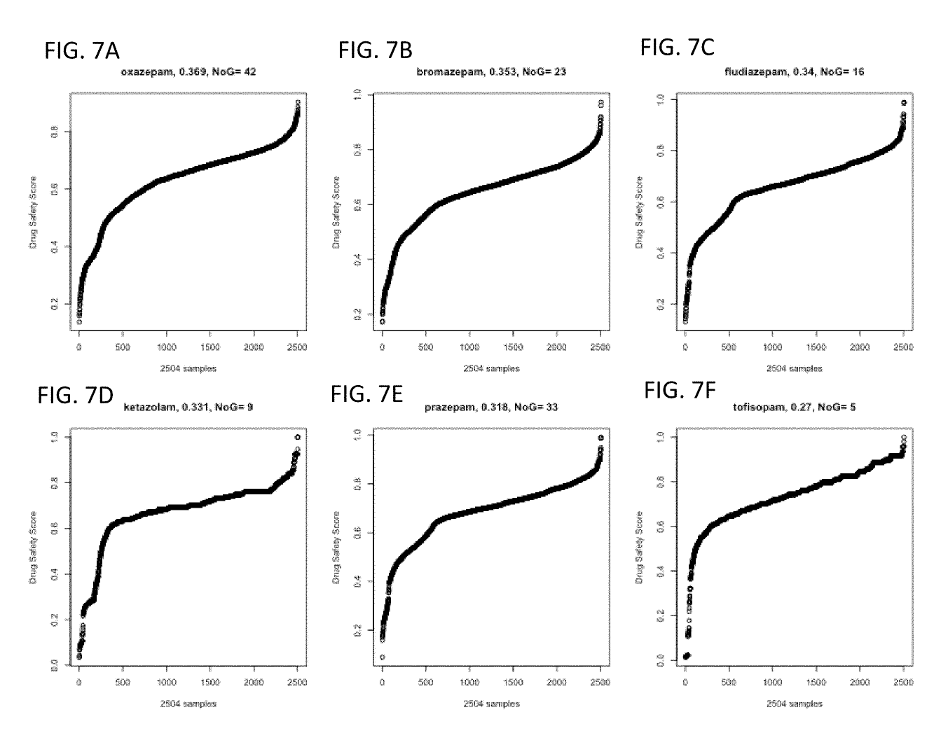

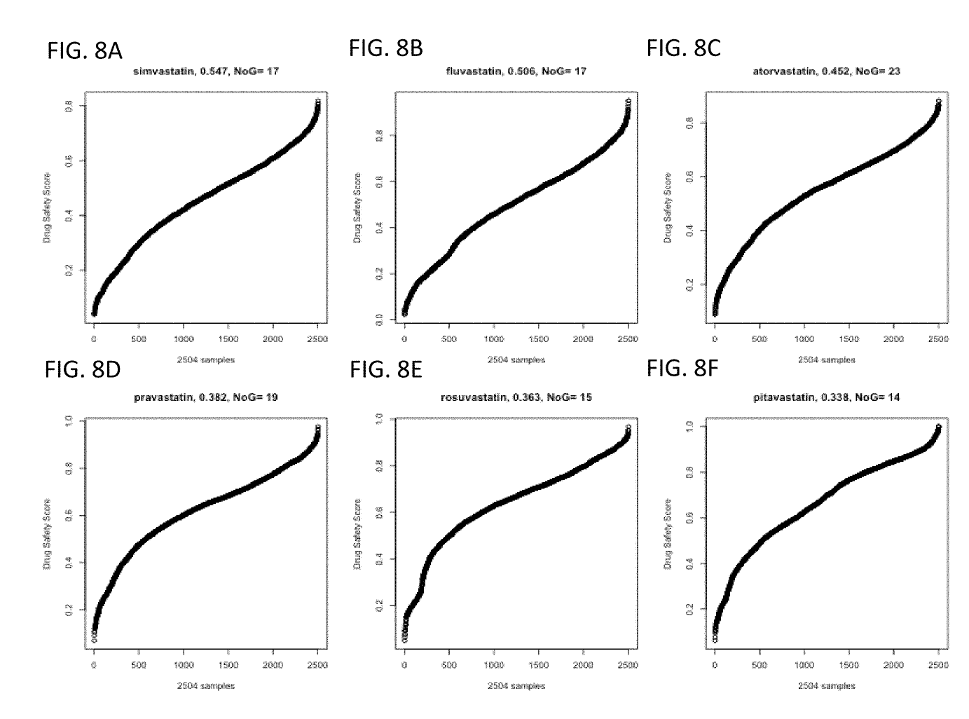

Individuals can have different response to different drugs within the same drug group. For example, as demonstrated in FIGS. 7A-F, N05BA drugs (benzodiazepine derivatives), classified as antipsychotics by the Anatomical Therapeutic Chemical (ACT) Classification, show different individual drug safety score distribution patterns. As presented in FIGS. 8A-F, C10AA drugs (HMG CoA reductase inhibitors), classified as lipid modifying agents by the Anatomical Therapeutic Chemical (ACT) Classification System provided by the WHO, also showed different individual drug safety score distribution patterns.

These distribution curves of individual drug safety scores can be used for choosing the safest drug for a subject by identifying the subject's ranking within the individual drug safety score distribution curve for each drug. For example, a subject may be in a high-risk subpopulation for a first drug, but not in a high-risk subpopulation for a second drug. In such case, the subject can choose the first drug instead of the second drug.

The graphs can be also used for calculating a population drug safety score for the drug group. In some embodiments of the present invention, a mean of population drug safety scores of multiple drugs within a drug group can be calculated to evaluate safety of the drug group.

### 6.4.6. Others

Although the exemplary embodiments of the present invention have been described in detail, the scope of the right of the present invention is not limited thereto. Various modifications and improvements made by those skilled in the art using the basic concept of the present invention defined in the appended claims are also included in the scope of the right of the present invention.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

The present disclosure provides computer-implemented methods and systems for evaluating safety of a drug or a drug group by performing certain computations associated with gene sequence variation information of individuals within a population. While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A computer-implemented method for evaluating safety of a drug, comprising the steps of:
obtaining, by an evaluation system, gene sequence variation information for each of a plurality of individuals within a population, wherein the gene sequence variation information is related to one or more genes associated with pharmacodynamics or pharmacokinetics of the drug;
calculating, by the evaluation system, a protein damage score for each of the plurality of individuals within the population using the gene sequence variation information;
calculating, by the evaluation system, an individual drug safety score for each of the plurality of individuals within the population based on the protein damage score to generate a set of individual drug safety scores; and
determining, by the evaluation system, safety of the drug for the population by identifying individuals having an individual drug safety score below or above a threshold value (T),
wherein the threshold value (T) is calculated by the Equation:
$$T = μ - κ \sqrt{\frac{1}{n} {\sum }_{i = 1}^{n} {( {d}_{i} - μ )}^{2}}$$
wherein T is a rational number satisfying 0<T<1, dᵢ is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores.

2. The method of claim 1, wherein the step of determining safety of the drug comprises: obtaining a curve representing the set of individual drug safety scores.

3. The method of claim 2, wherein the step of determining safety of the drug further comprises: calculating an area under the curve (AUC), a standardized area under the curve (S-AUC), an area upper the curve (AUPC), or a standardized area upper the curve (S-AUPC).

4. The method of claim 2, further comprising the step of calculating a population drug safety score using the following Equation:
$${S}_{p} ( d 1 , … , dn ) = \frac{1}{N} ( {AUC}_{d} ) = 1 - \frac{1}{N} ( {AUPC}_{d} ) ,$$
wherein Sp is the population drug safety score for the population, d₁₋ₙ is an individual drug safety score of an i-th individual (from 1 to n) within the population, AUC_{d} is the area under the curve for the drug d, AUPC_{d} is the area upper the curve for the drug d, and N or n is the number of individuals within the population.

5. The method of claim 2, wherein the threshold value (T) is determined based on the shape of the curve.

6. The method of claim 5, wherein the threshold value (T) is calculated based on the change in the slope of the curve.

7. The method of claim 2, wherein the threshold value (T) is determined by comparing the curve with a different curve corresponding to a different drug having similar pharmacodynamics or pharmacokinetics or a different drug previously identified to be unsafe.

8. The method of claim 1, wherein the threshold value (T) ranges from 0.1 to 0.5, from 0.2 to 0.4, or from 0.25 to 0.35, or is 0.3.

9. The method of claim 1, further comprising the step of providing a list of the individuals having an individual drug safety score below a threshold value or above a threshold value.

10. The method of claim 1, wherein the step of determining safety of the drug further comprises: calculating the number or the ratio of individuals having an individual drug safety score below the threshold value within the population.

11. The method of claim 1, further comprising the step of calculating a population drug safety score of the population, wherein the population drug safety score is related to the number or the ratio of individuals having a drug safety score below the threshold value within the population.

12. The method of claim 1, wherein the step of determining safety of the drug comprises: calculating a mean of individual drug safety scores of multiple individuals within the population, wherein the mean is calculated using one or more algorithms selected from the group consisting of a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication, a weighted multiplication, or a combination thereof.

13. The method of claim 12, further comprising the step of providing a population drug safety score of the population calculated by the following Equation:
$${S}_{p} ( d 1 , … , dn ) = \frac{1}{N} ( {\sum }_{H = 1}^{N} {S}_{d} ) ,$$
wherein Sp is the population drug safety score, dᵢ or Sd is an individual drug safety score of an individual within the population (i is from 1 to n), and n or N is the number of individuals within the population for which an individual drug safety score is obtained.

14. The method of claim 1, wherein the gene sequence variation information is information related to substitution, addition, or deletion of a nucleotide within the exon of the gene.

15. The method of claim 14, wherein the substitution, addition, or deletion of the nucleotide results from breakage, deletion, duplication, inversion or translocation of a chromosome.

16. The method of claim 1, further comprising the step of obtaining a gene sequence variation score from the gene sequence variation information, using one or more algorithm selected from the group consisting of: SIFT (Sorting Intolerant From Tolerant), PolyPhen, PolyPhen-2 (Polymorphism Phenotyping), MAPP (Multivariate Analysis of Protein Polymorphism), Logre (Log R Pfam E-value), Mutation Assessor, Condel, GERP (Genomic Evolutionary Rate Profiling), CADD (Combined Annotation-Dependent Depletion), MutationTaster, MutationTaster2, PROVEAN, PMuit, CEO (Combinatorial Entropy Optimization), SNPeffect, fathmm, MSRV (Multiple Selection Rule Voting), Align-GVGD, DANN, Eigen, KGGSeq, LRT (Likelihood Ratio Test), MetaLR, MetaSVM, MutPred, PANTHER, Parepro, phastCons, PhD-SNP, phyloP, PON-P, PON-P2, SiPhy, SNAP, SNPs&GO, VEP (Variant Effect Predictor), VEST (Variant Effect Scoring Tool), SNAP2, CAROL, PaPI, Grantham, SInBaD, VAAST, REVEL, CHASM (Cancer-specific High-throughput Annotation of Somatic Mutations), mCluster, nsSNPAnayzer, SAAPpred, HanSa, CanPredict, FIS and BONGO (Bonds ON Graphs).

17. The method of claim 16, wherein the gene sequence variation score is used to calculate the protein damage score or the individual drug safety score.

18. The method of claim 1, further comprising the step of obtaining a plurality of gene sequence variation scores from the gene sequence variation information, wherein the gene sequence variation information relates to substitution, addition, or deletion of a plurality of nucleotides within the gene.

19. The method of claim 18, wherein the protein damage score is calculated as a mean of the plurality of gene sequence variation scores.

20. The method of claim 19, wherein the mean is calculated using one or more algorithms selected from the group consisting of: a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication and a weighted multiplication.

21. The method of claim 18, wherein the protein damage score is calculated by the following Equation:
$${S}_{g} ( {υ}_{1 , … ,} {υ}_{n} ) = {( \frac{1}{n} {\sum }_{i = 1}^{n} {υ}_{i}^{p} )}^{\frac{1}{p}} ,$$
wherein S_{g} is a protein damage score of a protein encoded by the gene g, n is the number of the plurality of nucleotides corresponding to the plurality of gene sequence variation scores, vᵢ is a gene sequence variation score corresponding to an i-th gene sequence variation, and p is a non-zero real number.

22. The method of claim 18, wherein the protein damage score is calculated by the following Equation:
$${S}_{g} ( {υ}_{1 , … ,} {υ}_{n} ) = {( {\prod }_{i = 1}^{n} {υ}_{i}^{{w}_{i}} )}^{1 / {\sum }_{i = {1}^{{w}_{i}}}^{n}} ,$$
wherein S_{g} is a protein damage score of a protein encoded by the gene g, n is the number the plurality of nucleotides corresponding to the plurality of gene sequence variation scores, vᵢ is a gene sequence variation score corresponding to an i-th gene sequence variation, and wᵢ is a weighting assigned to the gene sequence variation score vᵢ of the i-th gene sequence variation.

23. The method of claim 1, further comprising the step of obtaining protein damage scores, wherein each of the protein damage scores corresponds to each of the plurality of proteins involved in the pharmacodynamics or pharmacokinetics of the drug.

24. The method of claim 23, wherein the individual drug safety score is calculated as a mean of the protein damage scores.

25. The method of claim 24, wherein the mean is calculated using one or more algorithm selected from the group consisting of: a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication and a weighted multiplication.

26. The method of claim 23, wherein the individual drug safety score is calculated by the following Equation:
$${S}_{d} ( {g}_{1 , … ,} {g}_{n} ) = {( \frac{1}{n} {\sum }_{i = 1}^{n} {g}_{i}^{p} )}^{\frac{1}{p}} ,$$
wherein Sd is an individual drug safety score of a drug d, n is the number of proteins encoded by one or more genes involved in the pharmacodynamics or pharmacokinetics of the drug d, gi is a protein damage score of the protein encoded by one or more genes involved in the pharmacodynamics or pharmacokinetics of the drug d, and p is a non-zero real number.

27. The method of claim 23, wherein the individual drug safety score is calculated by the following Equation:
$${S}_{d} ( {g}_{1 , … ,} {g}_{n} ) = {( {\prod }_{i = 1}^{n} {g}_{i}^{{w}_{i}} )}^{1 / {\sum }_{i = {1}^{{w}_{i}}}^{n}} ,$$
wherein S_{d} is a drug score of the drug d, n is the number of proteins encoded by one or more genes involved in the pharmacodynamics or pharmacokinetics of the drug d, gᵢ is a protein damage score of the protein encoded by one or more genes involved in the pharmacodynamics or pharmacokinetics of the drug d, and wᵢ is a weighting assigned to the protein damage score gᵢ of the protein encoded by one or more genes involved in the pharmacodynamics or pharmacokinetics of the drug d.

28. A computer-implemented method of evaluating safety of a drug group, comprising the steps of:
identifying drugs that belong to the drug group;
obtaining a population drug safety score for each of the drugs, thereby generating a set of population drug safety scores, wherein the population drug safety score is calculated by the method of claim 11; and
analyzing the set of population drug safety scores.

29. The method of claim 28, further comprising the step of: determining an order of priority among the drugs based on the analysis.

30. The method of claim 28, wherein the step of analyzing the set of population drug safety scores comprises:
calculating a mean of the set of population drug safety scores, wherein the mean is calculated using one or more algorithms selected from the group consisting of a geometric mean, an arithmetic mean, a harmonic mean, an arithmetic-geometric mean, an arithmetic-harmonic mean, a geometric-harmonic mean, a Pythagorean mean, a Heronian mean, a contraharmonic mean, a root-mean-square deviation, a centroid mean, an interquartile mean, a quadratic mean, a truncated mean, a winsorized mean, a weighted mean, a weighted geometric mean, a weighted arithmetic mean, a weighted harmonic mean, a mean of a function, a power mean, a generalized f-mean, a percentile, a maximum value, a minimum value, a mode, a median, a mid-range, a measure of central tendency, a simple multiplication, a weighted multiplication, or a combination thereof.

31. The method of claim 28, wherein the step of identifying drugs that belong to the drug group is performed based on (i) known drug classification methods, (ii) symptoms known to be treatable by the drugs, (iii) a chemical property of the drugs, (iv) an absorption or excretion mechanism of the drugs, or (v) a target of the drugs.

32. A method of evaluating safety of a drug to a subject, comprising the steps of
obtaining gene sequence variation information of the subject, wherein the gene sequence variation information is related to one or more genes associated with pharmacodynamics or pharmacokinetics of the drug;
obtaining a protein damage score of the subject using the gene sequence variation information;
obtaining a subject drug safety score of the subject based on the protein damage score; and
determining safety of the drug for the subject by comparing the subject drug safety score with a threshold value (T), wherein the threshold value (T) is calculated by the Equation:
$$T = μ - κ \sqrt{\frac{1}{n} {\sum }_{i = 1}^{n} {( {d}_{i} - μ )}^{2}}$$
wherein dᵢ is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores.

33. The method of claim 32, wherein the step of determining safety of the drug to the subject comprises the step of: determining a position of the subject drug safety score within the set of individual drug safety scores.

34. The method of claim 32, wherein the step of determining safety of the drug to the subject comprises the steps of:
drawing a curve with the set of individual drug safety scores;
obtaining an area under the curve (AUC), a standardized area under the curve (S-AUC), an area upper the curve (AUPC), or a standardized area upper the curve (S-AUPC); and
comparing the subject drug safety score with the AUC, S-AUC, AUPC, or S-AUPC.

35. The method of claim 32, wherein the step of determining safety of the drug to the subject comprises the steps of:
obtaining a population drug safety score of the population calculated by the Equation:
$${S}_{d} ( {d}_{1 , … ,} {d}_{n} ) = \frac{1}{n} {\prod }_{i = 1}^{n} {d}_{i} ,$$
wherein Sd is the population drug safety score of the population, dᵢ is an individual drug safety score of an i-th individual within the population (from 1 to n), and n is the number of individuals within the population; and
comparing the subject drug safety score with the population drug safety score (Sd).

36. The method of claim 32, further comprising the step of prescribing the drug based on the safety of the drug to the subject.

37. A computer-readable medium comprising stored instructions, wherein the instructions when executed by a processor cause the processor to perform the method of claim 1.

38. The computer-readable medium of claim 37, wherein the instructions further cause the processor to provide a report related to safety of the drug, safety of the drug group or safety of the drug to the subject.

39. A system for evaluating safety of a drug, comprising:
the computer-readable medium of claim 38; and
an output unit providing the report about the safety of the drug.

40. The system of claim 39, wherein the output unit provides the report by email, SMS messaging, web posting, phone call, electronic messaging, uploading or downloading.

41. The system of claim 39, further comprising a database to search for or retrieve information about one or more genes associated with pharmacodynamics or pharmacokinetics of the drug.
